(19) United States
(12) Patent Application Publication
(10) Pub. No.: US 2025/0002946 A1
(43) Pub. Date: Jan. 2, 2025

(54) Methods And Compositions For Increasing Homology-Directed Repair

(71) Applicant: Regeneron Pharmaceuticals, Inc., Tarrytown, NY (US)

(72) Inventors: Wei Ting Chelsea Lee, New York, NY (US); Ciro Bonetti, New York, NY (US)

(21) Appl. No.: 18/758,836

(22) Filed: Jun. 28, 2024

Related U.S. Application Data
(60) Provisional application No. 63/511,361, filed on Jun. 30, 2023.

(51) Int. Cl. C12N 15/90; C12N 9/22; C12N 15/10; C12N 15/11

(52) U.S. Cl. CPC C12N 15/907; C12N 9/22; C12N 15/1082; C12N 15/11; C12N 2310/20

(57) ABSTRACT

Provided herein are combinations comprising CRISPR/Cas systems, CtBP-interacting protein, and inhibitor of 53BP1 for use in enhancing homology-directed repair of CRISPR/Cas-mediated cleavage of a target DNA by an exogenous donor nucleic acid. Also provided are methods of using such combinations to make a targeted genetic modification in a cell by homology-directed repair of CRISPR/Cas-mediated cleavage at a target genomic locus in the cell.

### CROSS-REFERENCE TO RELATED APPLICATIONS

This application claims the benefit of U.S. Application No. 63/511,361, filed Jun. 30, 2023, which is herein incorporated by reference in its entirety for all purposes.

### REFERENCE TO A SEQUENCE LISTING SUBMITTED AS AN XML FILE VIA EFS WEB

The Sequence Listing written in file 614574SEQLIST.xml is 76,084 bytes, was created on Jun. 27, 2024, and is hereby incorporated by reference.

### BACKGROUND

The development of CRISPR/Cas technology provides an efficient approach to introduce site-specific modifications in the mammalian genome, offering great potential to study and treat a wide range of genetic diseases. Currently, the most used CRISPR system in genome engineering uses a single-guide RNA (sgRNA) and a CRISPR-associated endonuclease (Cas9), which generate double-stranded breaks (DSBs) at the targeted sequence. The two major DSB repair pathways in mammalian cells are (i) the error-prone non-homologous end joining (NHEJ) and (ii) the faithful homology-directed repair (HDR), which is restricted to the S and G2 phases of the cell cycle and depends on the availability of repair template carrying the modifications to be introduced. Methods to enhance HDR would be useful to provide better and more efficient ways to perform precise genome editing.

### SUMMARY

Provided herein are combinations comprising CRISPR/Cas systems, CtBP-interacting protein, and inhibitor of 53BP1 for use in enhancing homology-directed repair of CRISPR/Cas-mediated cleavage of a target DNA by an exogenous donor nucleic acid. Also provided are methods of using such combinations to make a targeted genetic modification in a cell by homology-directed repair of CRISPR/Cas-mediated cleavage at a target genomic locus in the cell.

In one aspect, provided are methods for making a targeted genetic modification by homology-directed repair at a target genomic locus in a cell. Some such methods comprise administering to the cell: (a) a Clustered Regularly Interspaced Short Palindromic Repeats (CRISPR) associated (Cas) protein or a nucleic acid encoding the Cas protein; (b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus; (c) a fusion protein or a nucleic acid encoding the fusion protein, wherein the fusion protein comprises a CtBP-interacting protein (CtIP) fused to the adaptor protein; (d) an inhibitor of 53BP1 (i53) protein or a nucleic acid encoding the i53 protein; and (e) an exogenous donor nucleic acid comprising a 5′ homology arm that hybridizes to a 5′ target sequence at the target genomic locus and a 3′ homology arm that hybridizes to a 3′ target sequence at the target genomic locus, optionally wherein the 5′ homology arm and the 3′ homology arm flank an insert nucleic acid, wherein the Cas protein and the guide RNA form a complex, the Cas protein cleaves the guide RNA target sequence to create a double-strand break, and the exogenous donor nucleic acid recombines with the target genomic locus via homology-directed repair to create the targeted genetic modification.

In some such methods, the Cas protein is administered to the cell in the form of a protein, optionally wherein the Cas protein is in a lipid nanoparticle. In some such methods, the nucleic acid encoding the Cas protein is administered to the cell, wherein the nucleic acid encoding the Cas protein comprises an RNA encoding the Cas protein, optionally wherein the RNA encoding the Cas protein is in a lipid nanoparticle. In some such methods, the nucleic acid encoding the Cas protein is administered to the cell, wherein the nucleic acid encoding the Cas protein comprises a DNA encoding the Cas protein, optionally wherein the DNA encoding the Cas protein is in a viral vector, optionally wherein the viral vector is a recombinant adeno-associated virus (AAV) vector. In some such methods, the Cas protein is a Cas9 protein. In some such methods, the Cas9 protein is a Streptococcus pyogenes Cas9 protein, a Campylobacter jejuni Cas9 protein, or a Staphylococcus aureus Cas9 protein, optionally wherein the Cas9 protein is the Streptococcus pyogenes Cas9 protein.

In some such methods, the guide RNA is administered in the form of RNA, optionally wherein the guide RNA is in a lipid nanoparticle. In some such methods, the one or more DNAs encoding the guide RNA are administered to the cell, optionally wherein the one or more DNAs encoding the guide RNA are in a viral vector, optionally wherein the viral vector is a recombinant AAV vector. In some such methods, the guide RNA comprises two adaptor-binding elements to which the adaptor protein can specifically bind. In some such methods, a first adaptor-binding element is within a first loop of the guide RNA, and a second adaptor-binding element is within a second loop of the guide RNA. In some such methods, the guide RNA is a single guide RNA comprising a CRISPR RNA (crRNA) portion fused to a transactivating CRISPR RNA (tracrRNA) portion, and wherein the first loop is the tetraloop corresponding to residues 13-16 of SEQ ID NO: 11, 13, 15, or 16, and the second loop is the stem loop 2 corresponding to residues 53-56 of SEQ ID NO: 11, 13, 15, or 16. In some such methods, the adaptor-binding element comprises the sequence set forth in SEQ ID NO: 19 or 20. In some such methods, the guide RNA comprises the sequence set forth in SEQ ID NO: 21, 22, 23, 24, 25, or 26.

In some such methods, the fusion protein is administered to the cell in the form of a protein, optionally wherein the fusion protein is in a lipid nanoparticle. In some such methods, the nucleic acid encoding the fusion protein is administered to the cell, wherein the nucleic acid encoding the fusion protein comprises an RNA encoding the fusion protein, optionally wherein the RNA encoding the fusion protein is in a lipid nanoparticle. In some such methods, the nucleic acid encoding the fusion protein is administered to the cell, wherein the nucleic acid encoding the fusion protein comprises a DNA encoding the fusion protein, optionally wherein the DNA encoding the fusion protein is in a viral vector, optionally wherein the viral vector is a recombinant AAV vector. In some such methods, the adaptor protein comprises an MS2 coat protein or a functional fragment or variant thereof. In some such methods, the adaptor protein comprises a sequence at least 90%, 95%, 96%, 97%, 98%, 99%, or 100% identical to the sequence set forth in SEQ ID NO: 32. In some such methods, the adaptor protein is encoded by a sequence at least 90%, 95%, 96%, 97%, 98%, 99%, or 100% identical to the sequence set forth in SEQ ID NO: 33. In some such methods, the CtIP protein comprises a sequence at least 90%, 95%, 96%, 97%, 98%, 99%, or 100% identical to the sequence set forth in SEQ ID NO: 34. In some such methods, the CtIP protein is encoded by a sequence at least 90%, 95%, 96%, 97%, 98%, 99%, or 100% identical to the sequence set forth in SEQ ID NO: 36. In some such methods, the fusion protein comprises a sequence at least 90%, 95%, 96%, 97%, 98%, 99%, or 100% identical to the sequence set forth in SEQ ID NO: 30. In some such methods, the fusion protein is encoded by a sequence at least 90%, 95%, 96%, 97%, 98%, 99%, or 100% identical to the sequence set forth in SEQ ID NO: 31.

In some such methods, the i53 protein is administered to the cell in the form of a protein, optionally wherein the i53 protein is in a lipid nanoparticle. In some such methods, the nucleic acid encoding the i53 protein is administered to the cell, wherein the nucleic acid encoding the i53 protein comprises an RNA messenger RNA encoding the i53 protein, optionally wherein the RNA encoding the i53 protein is in a lipid nanoparticle. In some such methods, the nucleic acid encoding the i53 protein is administered to the cell, wherein the nucleic acid encoding the i53 protein comprises a DNA encoding the i53 protein, optionally wherein the DNA encoding the i53 protein is in a viral vector, optionally wherein the viral vector is a recombinant AAV vector. In some such methods, the i53 protein comprises a sequence at least 90%, 95%, 96%, 97%, 98%, 99%, or 100% identical to the sequence set forth in SEQ ID NO: 40 or 42. In some such methods, the i53 protein is encoded by a sequence at least 90%, 95%, 96%, 97%, 98%, 99%, or 100% identical to the sequence set forth in SEQ ID NO: 41 or 43.

In some such methods, the exogenous donor nucleic acid comprises the insert nucleic acid. In some such methods, the exogenous donor nucleic acid is in a viral vector. In some such methods, the viral vector is a recombinant AAV vector. In some such methods, the exogenous donor nucleic acid is a large targeting vector (LTVEC), wherein: (a) the LTVEC is at least 10 kb; (b) the sum total of the 5′ and 3′ homology arms of the LTVEC is at least 10 kb; (c) the LTVEC is from about 50 kb to about 300 kb; or (d) the sum total of the 5′ and 3′ homology arms of the LTVEC is from about 10 kb to about 200 kb.

In some such methods, the nucleic acid encoding the fusion protein is administered to the cell, wherein the nucleic acid encoding the fusion protein comprises an RNA encoding the fusion protein, wherein the nucleic acid encoding the i53 protein is administered to the cell, the nucleic acid encoding the i53 protein comprises an RNA encoding the i53 protein, and the RNA encoding the fusion protein and the RNA encoding the i53 protein are in a lipid nanoparticle. In some such methods, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the nucleic acid encoding the fusion protein is administered to the cell, wherein the nucleic acid encoding the fusion protein comprises an RNA encoding the fusion protein, the nucleic acid encoding the i53 protein is administered to the cell, wherein the nucleic acid encoding the i53 protein comprises an RNA encoding the i53 protein, and the RNA encoding the fusion protein and the RNA encoding the i53 protein are in a lipid nanoparticle.

In some such methods, the guide RNA comprises two adaptor-binding elements to which the adaptor protein can specifically bind, wherein a first adaptor-binding element is within a first loop of the guide RNA, and a second adaptor-binding element is within a second loop of the guide RNA, wherein the adaptor protein comprises an MS2 coat protein or a functional fragment or variant thereof, wherein the nucleic acid encoding the Cas protein is administered to the cell, wherein the nucleic acid encoding the Cas protein comprises an RNA encoding the Cas protein, wherein the nucleic acid encoding the fusion protein is administered to the cell, wherein the nucleic acid encoding the fusion protein comprises an RNA encoding the fusion protein, wherein the nucleic acid encoding the i53 protein is administered to the cell, wherein the nucleic acid encoding the i53 protein comprises an RNA encoding the i53 protein, wherein the RNA encoding the Cas protein, the RNA encoding the fusion protein, and the RNA encoding the i53 protein are in a lipid nanoparticle, wherein the one or more DNAs encoding the guide RNA are administered to the cell, and wherein the one or more DNAs encoding the guide RNA and the exogenous donor nucleic acid are in a recombinant AAV vector.

In some such methods, the guide RNA comprises two adaptor-binding elements to which the adaptor protein can specifically bind, wherein a first adaptor-binding element is within a first loop of the guide RNA, and a second adaptor-binding element is within a second loop of the guide RNA, wherein the adaptor protein comprises an MS2 coat protein or a functional fragment or variant thereof, wherein the nucleic acid encoding the Cas protein is administered to the cell, wherein the nucleic acid encoding the Cas protein comprises an RNA encoding the Cas protein, wherein the nucleic acid encoding the fusion protein is administered to the cell, wherein the nucleic acid encoding the fusion protein comprises an RNA encoding the fusion protein, wherein the nucleic acid encoding the i53 protein is administered to the cell, wherein the nucleic acid encoding the i53 protein comprises an RNA encoding the i53 protein, wherein the guide RNA is administered to the cell in the form of RNA, wherein the RNA encoding the Cas protein, the RNA encoding the fusion protein, the RNA encoding the i53 protein, and the guide RNA are in a lipid nanoparticle, wherein the exogenous donor nucleic acid is in a recombinant AAV vector.

In some such methods, the cell is a mammalian cell. In some such methods, the cell is a rodent cell. In some such methods, the cell is a mouse cell or a rat cell. In some such methods, the cell is a mouse cell. In some such methods, the cell is a human cell. In some such methods, the cell is in vitro. In some such methods, the cell is in vivo.

In another aspect, provided are compositions or combinations. Some such compositions or combinations comprise (a) a Clustered Regularly Interspaced Short Palindromic Repeats (CRISPR) associated (Cas) protein or a nucleic acid encoding the Cas protein; (b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus; (c) a fusion protein or a nucleic acid encoding the fusion protein, wherein the fusion protein comprises a CtBP-interacting protein (CtIP) fused to the adaptor protein; (d) an inhibitor of 53BP1 (i53) protein or a nucleic acid encoding the i53 protein; and (e) an exogenous donor nucleic acid comprising a 5′ homology arm that hybridizes to a 5′ target sequence at the target genomic locus and a 3′ homology arm that hybridizes to a 3′ target sequence at the target genomic locus, optionally wherein the 5′ homology arm and the 3′ homology arm flank an insert nucleic acid.

In some such compositions or combinations, the composition or combination comprises the Cas protein is in the form of a protein, optionally wherein the Cas protein is in a lipid nanoparticle. In some such compositions or combinations, the composition or combination comprises the nucleic acid encoding the Cas protein, wherein the nucleic acid encoding the Cas protein comprises an RNA encoding the Cas protein, optionally wherein the RNA encoding the Cas protein is in a lipid nanoparticle. In some such compositions or combinations, the composition or combination comprises the nucleic acid encoding the Cas protein, wherein the nucleic acid encoding the Cas protein comprises a DNA encoding the Cas protein, optionally wherein the DNA encoding the Cas protein is in a viral vector, optionally wherein the viral vector is a recombinant adeno-associated virus (AAV) vector. In some such compositions or combinations, the Cas protein is a Cas9 protein. In some such compositions or combinations, the Cas9 protein is a Streptococcus pyogenes Cas9 protein, a Campylobacter jejuni Cas9 protein, or a Staphylococcus aureus Cas9 protein, optionally wherein the Cas9 protein is the Streptococcus pyogenes Cas9 protein.

In some such compositions or combinations, the composition or combination comprises the guide RNA in the form of RNA, optionally wherein the guide RNA is in a lipid nanoparticle. In some such compositions or combinations, the composition or combination comprises the one or more DNAs encoding the guide RNA, optionally wherein the one or more DNAs encoding the guide RNA are in a viral vector, optionally wherein the viral vector is a recombinant AAV vector. In some such compositions or combinations, the guide RNA comprises two adaptor-binding elements to which the adaptor protein can specifically bind. In some such compositions or combinations, a first adaptor-binding element is within a first loop of the guide RNA, and a second adaptor-binding element is within a second loop of the guide RNA. In some such compositions or combinations, the guide RNA is a single guide RNA comprising a CRISPR RNA (crRNA) portion fused to a transactivating CRISPR RNA (tracrRNA) portion, and wherein the first loop is the tetraloop corresponding to residues 13-16 of SEQ ID NO: 11, 13, 15, or 16, and the second loop is the stem loop 2 corresponding to residues 53-56 of SEQ ID NO: 11, 13, 15, or 16. In some such compositions or combinations, the adaptor-binding element comprises the sequence set forth in SEQ ID NO: 19 or 20. In some such compositions or combinations, the guide RNA comprises the sequence set forth in SEQ ID NO: 21, 22, 23, 24, 25, or 26.

In some such compositions or combinations, the composition or combination comprises the fusion protein in the form of a protein, optionally wherein the fusion protein is in a lipid nanoparticle. In some such compositions or combinations, the composition or combination comprises the nucleic acid encoding the fusion protein, wherein the nucleic acid encoding the fusion protein comprises an RNA encoding the fusion protein, optionally wherein the RNA encoding the fusion protein is in a lipid nanoparticle. In some such compositions or combinations, the composition or combination comprises the nucleic acid encoding the fusion protein, wherein the nucleic acid encoding the fusion protein comprises a DNA encoding the fusion protein, optionally wherein the DNA encoding the fusion protein is in a viral vector, optionally wherein the viral vector is a recombinant AAV vector. In some such compositions or combinations, the adaptor protein comprises an MS2 coat protein or a functional fragment or variant thereof. In some such compositions or combinations, the adaptor protein comprises a sequence at least 90%, 95%, 96%, 97%, 98%, 99%, or 100% identical to the sequence set forth in SEQ ID NO: 32. In some such compositions or combinations, the adaptor protein is encoded by a sequence at least 90%, 95%, 96%, 97%, 98%, 99%, or 100% identical to the sequence set forth in SEQ ID NO: 33. In some such compositions or combinations, the CtIP protein comprises a sequence at least 90%, 95%, 96%, 97%, 98%, 99%, or 100% identical to the sequence set forth in SEQ ID NO: 34. In some such compositions or combinations, the CtIP protein is encoded by a sequence at least 90%, 95%, 96%, 97%, 98%, 99%, or 100% identical to the sequence set forth in SEQ ID NO: 36. In some such compositions or combinations, the fusion protein comprises a sequence at least 90%, 95%, 96%, 97%, 98%, 99%, or 100% identical to the sequence set forth in SEQ ID NO: 30. In some such compositions or combinations, the fusion protein is encoded by a sequence at least 90%, 95%, 96%, 97%, 98%, 99%, or 100% identical to the sequence set forth in SEQ ID NO: 31.

In some such compositions or combinations, the composition or combination comprises the i53 protein in the form of a protein, optionally wherein the i53 protein is in a lipid nanoparticle. In some such compositions or combinations, the composition or combination comprises the nucleic acid encoding the i53 protein, wherein the nucleic acid encoding the i53 protein comprises an RNA messenger RNA encoding the i53 protein, optionally wherein the RNA encoding the i53 protein is in a lipid nanoparticle. In some such compositions or combinations, the composition or combination comprises the nucleic acid encoding the i53 protein, wherein the nucleic acid encoding the i53 protein comprises a DNA encoding the i53 protein, optionally wherein the DNA encoding the i53 protein is in a viral vector, optionally wherein the viral vector is a recombinant AAV vector. In some such compositions or combinations, the i53 protein comprises a sequence at least 90%, 95%, 96%, 97%, 98%, 99%, or 100% identical to the sequence set forth in SEQ ID NO: 40 or 42. In some such compositions or combinations, the i53 protein is encoded by a sequence at least 90%, 95%, 96%, 97%, 98%, 99%, or 100% identical to the sequence set forth in SEQ ID NO: 41 or 43.

In some such compositions or combinations, the exogenous donor nucleic acid comprises the insert nucleic acid. In some such compositions or combinations, the exogenous donor nucleic acid is in a viral vector. In some such compositions or combinations, the viral vector is a recombinant AAV vector. In some such compositions or combinations, the exogenous donor nucleic acid is a large targeting vector (LTVEC), wherein: (a) the LTVEC is at least 10 kb; (b) the sum total of the 5′ and 3′ homology arms of the LTVEC is at least 10 kb; (c) the LTVEC is from about 50 kb to about 300 kb; or (d) the sum total of the 5′ and 3′ homology arms of the LTVEC is from about 10 kb to about 200 kb.

In some such compositions or combinations, the composition or combination comprises the nucleic acid encoding the fusion protein, wherein the nucleic acid encoding the fusion protein comprises an RNA encoding the fusion protein, the composition or combination comprises the nucleic acid encoding the i53 protein, wherein the nucleic acid encoding the i53 protein comprises an RNA encoding the i53 protein, and the RNA encoding the fusion protein and the RNA encoding the i53 protein are in a lipid nanoparticle. In some such compositions or combinations, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises the nucleic acid encoding the fusion protein, wherein the nucleic acid encoding the fusion protein comprises an RNA encoding the fusion protein, the composition or combination comprises the nucleic acid encoding the i53 protein, wherein the nucleic acid encoding the i53 protein comprises an RNA encoding the i53 protein, and the RNA encoding the fusion protein and the RNA encoding the i53 protein are in a lipid nanoparticle.

In some such compositions or combinations, the guide RNA comprises two adaptor-binding elements to which the adaptor protein can specifically bind, wherein a first adaptor-binding element is within a first loop of the guide RNA, and a second adaptor-binding element is within a second loop of the guide RNA, wherein the adaptor protein comprises an MS2 coat protein or a functional fragment or variant thereof, wherein the composition or combination comprises the nucleic acid encoding the Cas protein, wherein the nucleic acid encoding the Cas protein comprises an RNA encoding the Cas protein, wherein the composition or combination comprises the nucleic acid encoding the fusion protein, wherein the nucleic acid encoding the fusion protein comprises an RNA encoding the fusion protein, wherein the composition or combination comprises the nucleic acid encoding the i53 protein, wherein the nucleic acid encoding the i53 protein comprises an RNA encoding the i53 protein, wherein the RNA encoding the Cas protein, the RNA encoding the fusion protein, and the RNA encoding the i53 protein are in a lipid nanoparticle, wherein the composition or combination comprises the one or more DNAs encoding the guide RNA, and wherein the one or more DNAs encoding the guide RNA and the exogenous donor nucleic acid are in a recombinant AAV vector.

In some such compositions or combinations, the guide RNA comprises two adaptor-binding elements to which the adaptor protein can specifically bind, wherein a first adaptor-binding element is within a first loop of the guide RNA, and a second adaptor-binding element is within a second loop of the guide RNA, wherein the adaptor protein comprises an MS2 coat protein or a functional fragment or variant thereof, wherein the composition or combination comprises the nucleic acid encoding the Cas protein, wherein the nucleic acid encoding the Cas protein comprises an RNA encoding the Cas protein, wherein the composition or combination comprises the nucleic acid encoding the fusion protein, wherein the nucleic acid encoding the fusion protein comprises an RNA encoding the fusion protein, wherein the composition or combination comprises the nucleic acid encoding the i53 protein, wherein the nucleic acid encoding the i53 protein comprises an RNA encoding the i53 protein, wherein the composition or combination comprises the guide RNA is in the form of RNA, wherein the RNA encoding the Cas protein, the RNA encoding the fusion protein, the RNA encoding the i53 protein, and the guide RNA are in a lipid nanoparticle, wherein the exogenous donor nucleic acid is in a recombinant AAV vector.

### BRIEF DESCRIPTION OF THE FIGURES

The patent application file contains at least one drawing executed in color. Copies of this patent application with color drawing(s) will be provided by the Office upon request and payment of the necessary fec.

FIG. 1 shows a schematic for insertion of a CRISPR-mediated homology-directed repair (HDR) reporter targeting the LMNA gene. In this assay, the coding sequence for the mClover fluorescent protein is designed to be integrated into the 5′ end of the Lamin A (LMNA) gene by HDR. The resulting expression and distinct localization of the mClover-LMNA fusion protein can then be visualized and quantified by microscopy. An AAV2 vector (AAVsgLMNA+mClover) expressing sgRNA that targets LMNA (sgLMNA) and containing mClover sequence flanked by appropriate homology arms (HAs) was used as the HDR donor template. The donor template is flanked by sgLMNA recognizing sequence, which allows for donor linearization upon sgLMNA expression in cell.

FIGS. 2A-2C show evaluation of baseline HDR in Cas9-expressing HEK293 cells with increasing MOI of the AAV2 HDR template using the assay shown in FIG. 1. FIG. 2A shows mClover and Hoechst staining for assessing the percentage of mClover-LMNA-positive cells. FIG. 2B shows the HDR efficiency as measured by the percentage of mClover-LMNA-positive cells. FIG. 2C shows data from negative controls, including co-treatment with mirin or using a donor template without homology arms.

FIGS. 3A-3B show screening potential HDR-boosting factors for selectively favoring HDR repair at CRISPR/Cas9-mediated double-strand breaks in Cas9-expressing HEK293 cells. FIG. 3A shows a schematic for recruiting potential HDR-boosting factors to Cas9 cleavage sites using the MS2-tagging approach. In this approach, the scaffold sequence of an sgRNA is modified to include MS2 phage aptamers (sgRNA2.0), and the HDR-boosting proteins are fused to the MS2 coat protein (MS2) for interaction with sgRNA2.0. FIG. 3B shows the effects on HDR efficiency as measured by the percentage of mClover-LMNA-positive cells using the assay shown in FIG. 1.

FIGS. 4A-4C show the effects of MS2-CtIP mRNA on HDR efficiency in Cas9-expressing HEK293 cells at increasing amount of MS2-CtIP mRNA packaged into LNP, demonstrating that CtIP-mediated DNA end resection promotes cellular commitment to HDR. FIG. 4A shows mClover and Hoechst staining for assessing the percentage of mClover-LMNA-positive cells. FIG. 4B shows the HDR efficiency as measured by the percentage of mClover-LMNA-positive cells as well as the percentage of cells with unwanted insertions/deletions (indels) caused by repair via non-homologous end joining. All “HR efficiency” plots show the absolute % mCLOVER cells from 3 experimental replicates. The box extends from the 25th to 75th percentiles. The line in the middle of the box is plotted at the median. The “+” is plotted at the mean. The whiskers go down to the smallest value and up to the largest. To ensure consistency, all microscopy images were taken from the same replicate (1 of 3 experimental replicates) and the 9% mCLOVER from each condition is shown in bottom right of the respective image. FIG. 4C shows the effects on HDR efficiency of MS2-CtIP using sgRNA containing MS2-binding loops (2.0) or sgRNA containing no MS2-binding loops (REG).

FIG. 5 shows a comparison of HDR efficiency as measured by the percentage of mClover-LMNA-positive cells when using plasmid delivery of MS2-CtIP or LNP delivery of MS2-CtIP mRNA in Cas9-expressing HEK293 cells.

FIGS. 6A-6B show the effects of i53 mRNA on HDR efficiency in Cas9-expressing HEK293 cells at increasing amount of i53 mRNA packaged into LNP, demonstrating that 53BP1 inhibition provides a pro-resection environment at double-strand breaks and further promotes HDR. FIG. 6A shows mClover and Hoechst staining for assessing the percentage of mClover-LMNA-positive cells. FIG. 6B shows the HDR efficiency as measured by the percentage of mClover-LMNA-positive cells as well as the percentage of cells with unwanted insertions/deletions (indels) caused by repair via non-homologous end joining. All “HR efficiency” plots show the absolute % mCLOVER cells from 3 experimental replicates. The box extends from the 25th to 75th percentiles. The line in the middle of the box is plotted at the median. The “+” is plotted at the mean. The whiskers go down to the smallest value and up to the largest. To ensure consistency, all microscopy images were taken from the same replicate (1 of 3 experimental replicates) and the 9% mCLOVER from each condition is shown in bottom right of the respective image.

FIG. 7 shows the combinatorial effects of MS2-CtIP and i53 on HDR efficiency enhancement in Cas9-expressing HEK293 cells, demonstrating that the co-expression of i53 and MS2-CtIP significantly increase CRISPR-stimulated HDR. All “HR efficiency” plots show the absolute % mCLOVER cells from 3 experimental replicates. The box extends from the 25th to 75th percentiles. The line in the middle of the box is plotted at the median. The “+” is plotted at the mean. The whiskers go down to the smallest value and up to the largest. To ensure consistency, all microscopy images were taken from the same replicate (1 of 3 experimental replicates) and the % mCLOVER from each condition is shown in bottom right of the respective image.

FIG. 8 shows the combinatorial effects of MS2-CtIP and i53 on HDR efficiency in Cas9-expressing HEK293 cells versus the effect of AZD7648, a DNA-PKcs inhibitor.

FIGS. 9A-9B shows the combinatorial effects of MS2-CtIP and i53 on HDR efficiency in HEK293 cells at increasing doses of AAV_{sgLMNA+mClover}. Two LNPs were used: LNP_{booster} which encapsulates Cas9, MS2-CtIP, and i53 mRNAs, and LNP_{baseline} which encapsulates Cas9 and mCherry mRNAs. In FIG. 9A, the LNPs were transfected into HEK293 cells that were transduced with different MOIs of AAV_{sgLMNA+mClover}. In FIG. 9B, the experiment was repeated using guide RNAs targeting the C-terminus of two other genes, HMGA1 and SEC61B.

FIG. 10 shows the combinatorial effects of MS2-CtIP and i53 on HDR efficiency in Cas9-expressing HEK293 cells in the context of non-viral donor delivery, specifically linear closed-ended dsDNA containing mClover coding sequence flanked by LMNA HA sequences as described above (dsDNA_{mclover-LMNA}) delivered via electroporation together with plasmid encoding sgLMNA2.0.

FIG. 11 shows the combinatorial effects of MS2-CtIP and i53 on HDR efficiency in Cas9-expressing HEK293 cells in the context of non-viral donor delivery, specifically linear closed-ended dsDNA containing mClover coding sequence flanked by LMNA HA sequences as described above (dsDNA_{mclover-LMNA}) delivered via electroporation together with sgLMNA2.0 delivered in the form of RNA.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous Rosa26 sequence of an animal refers to a native Rosa26 sequence that naturally occurs at the Rosa26 locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “Rosa26 locus” may refer to the specific location of a Rosa26 gene, Rosa26 DNA sequence, or Rosa26 position on a chromosome of the genome of an organism that has been identified as to where such a sequence resides. A “Rosa26 locus” may comprise a regulatory element of a Rosa26 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p<0.05.

In the event of a conflict between a sequence in the application and an indicated accession number or position in an accession number, the sequence in the application predominates.

### DETAILED DESCRIPTION

### 1. Overview

Provided herein are combinations comprising CRISPR/Cas systems, CtBP-interacting protein (CtIP), and inhibitor of 53BP1 for use in enhancing homology-directed repair of CRISPR/Cas-mediated cleavage of a target DNA by an exogenous donor nucleic acid. Also provided are methods of using such combinations to make a targeted genetic modification in a cell by homology-directed repair of CRISPR/Cas-mediated cleavage at a target genomic locus in the cell.

The compositions and methods disclosed herein improve precision CRISPR editing (precise gene knock-in) efficiency by stimulating DNA end resection, and thus homologous direct repair (HDR) efficiency, in CRISPR-targeted mammalian cells. These compositions and methods can be applied in non-cycling cells (not prone to HDR).

We observed that combining 53BP1 inhibition with CtIP localization leads to an additive or combinatorial enhancement in HDR efficiency. This outcome was not anticipated, as it was expected for their effects to be redundant. In addition, contrary to expectations of hyper-resection leading to mutagenic single-strand annealing repair, an expected consequence of 53BP1 inhibition and CtIP-mediated resection, we observed an increase in error-free HDR. Our approach allows for a stoichiometry of 1:4 for sgRNA2.0: MS2-CtIP, meanwhile providing multiple copies i53s as a global but transient blockade of 53BP1, ensuring their availabilities and activities to be synchronized to CRISPR activities. In some embodiments, we employ LNP-mediated mRNA delivery, ensuring transient expression of our HDR booster, thus minimizing long-term impacts of these factors on global genomic integrity.

### II. Methods, Compositions, and Combinations for Promoting Homology-Directed Repair

Provided herein are compositions or combinations for use in promoting homology-directed repair. Such compositions or combinations can comprise: (a) a Clustered Regularly Interspaced Short Palindromic Repeats (CRISPR) associated (Cas) protein or a nucleic acid encoding the Cas protein; (b) a guide RNA or one or more DNAs encoding the guide RNA, wherein the guide RNA is capable of forming a complex with the Cas protein and guiding it to a guide RNA target sequence at the target genomic locus; (c) a CtBP-interacting protein (CtIP) or a nucleic acid encoding the CtIP protein; (d) an inhibitor of 53BP1 (i53) protein or a nucleic acid encoding the i53 protein; and (e) an exogenous donor nucleic acid comprising a 5′ homology arm that hybridizes to a 5′ target sequence at the target genomic locus and a 3′ homology arm that hybridizes to a 3′ target sequence at the target genomic locus. Optionally, the 5′ homology arm and the 3′ homology arm flank an insert nucleic acid. For example, such compositions or combinations can comprise: (a) a Cas protein or a nucleic acid encoding the Cas protein; (b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus; (c) a fusion protein or a nucleic acid encoding the fusion protein, wherein the fusion protein comprises a CtIP protein fused to the adaptor protein; (d) an i53 protein or a nucleic acid encoding the i53 protein; and (c) an exogenous donor nucleic acid comprising a 5′ homology arm that hybridizes to a 5′ target sequence at the target genomic locus and a 3′ homology arm that hybridizes to a 3′ target sequence at the target genomic locus. Optionally, the 5′ homology arm and the 3′ homology arm flank an insert nucleic acid. As used herein, the term “in combination with” means that some components may be administered prior to, concurrent with, or after the administration of other components. The different components of the combination can be formulated into a single composition, e.g., for simultaneous delivery, or formulated separately into two or more compositions (e.g., a kit including each component, for example, wherein the further agent is in a separate formulation). Suitable CRISPR/Cas systems, including Cas proteins and guide RNAs, are described in more detail elsewhere herein. Likewise, suitable CtIP proteins, adaptor proteins, fusion proteins, i53 proteins, and exogenous donor nucleic acids are described in more detail elsewhere herein.

Likewise, provided herein are methods for making a targeted genetic modification by homology-directed repair of a double-strand break at a target genomic locus in a cell. Such methods can comprise administering to a cell: (a) a Cas protein or a nucleic acid encoding the Cas protein; (b) a guide RNA or one or more DNAs encoding the guide RNA, wherein the guide RNA is capable of forming a complex with the Cas protein and guiding it to a guide RNA target sequence at the target genomic locus; (c) a CtIP protein or a nucleic acid encoding the CtIP protein; (d) an i53 protein or a nucleic acid encoding the i53 protein; and (c) an exogenous donor nucleic acid comprising a 5′ homology arm that hybridizes to a 5′ target sequence at the target genomic locus and a 3′ homology arm that hybridizes to a 3′ target sequence at the target genomic locus, wherein the Cas protein and the guide RNA form a complex, the Cas protein cleaves the guide RNA target sequence to create a double-strand break, and the exogenous donor nucleic acid recombines with the target genomic locus via homology-directed repair to create the targeted genetic modification. Optionally, wherein the 5′ homology arm and the 3′ homology arm flank an insert nucleic acid. For example, such methods can comprise administering to a cell: (a) a Cas protein or a nucleic acid encoding the Cas protein; (b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus; (c) a fusion protein or a nucleic acid encoding the fusion protein, wherein the fusion protein comprises a CtIP protein fused to the adaptor protein; (d) an i53 protein or a nucleic acid encoding the i53 protein; and (e) an exogenous donor nucleic acid comprising a 5′ homology arm that hybridizes to a 5′ target sequence at the target genomic locus and a 3′ homology arm that hybridizes to a 3′ target sequence at the target genomic locus, wherein the Cas protein and the guide RNA form a complex, the Cas protein cleaves the guide RNA target sequence to create a double-strand break, and the exogenous donor nucleic acid recombines with the target genomic locus via homology-directed repair to create the targeted genetic modification. Optionally, the 5′ homology arm and the 3′ homology arm flank an insert nucleic acid. Suitable CRISPR/Cas systems, including Cas proteins and guide RNAs, are described in more detail elsewhere herein. Likewise, suitable CtIP proteins, adaptor proteins, fusion proteins, i53 proteins, and exogenous donor nucleic acids are described in more detail elsewhere herein.

The Cas protein in the composition or combination or methods can be any suitable Cas protein and can be in any form, such as in the form of a protein, in the form of an RNA encoding the Cas protein, or in the form of a DNA encoding the Cas protein (e.g., in a vector such as a recombinant adeno-associated virus (AAV) vector as described in more detail elsewhere herein). Likewise, the Cas protein or the nucleic acid encoding the Cas protein can be in any form for delivery, such as in a lipid nanoparticle as described in more detail elsewhere herein. The Cas protein can be any Cas protein described herein, such as a Cas9 protein. In a particular example, the Cas9 protein can be a Streptococcus pyogenes Cas9 protein, a Campylobacter jejuni Cas9 protein, or a Staphylococcus aureus Cas9 protein (e.g., a Streptococcus pyogenes Cas9 protein). Cas proteins and CRISPR/Cas systems are described in more detail elsewhere herein.

The guide RNA in the composition or combination or methods can be any suitable guide RNA, and can be in any form, such as in the form of an RNA or in the form of one or more DNAs encoding the guide RNA (e.g., in a vector such as a recombinant adeno-associated virus (AAV) vector as described in more detail elsewhere herein). Likewise, the guide RNA or the DNA or DNAs encoding the guide RNA can be in any form for delivery, such as in a lipid nanoparticle as described in more detail elsewhere herein. In one example, the guide RNA comprises two adaptor-binding elements to which the adaptor protein can specifically bind. For example, the guide RNA can comprise a first adaptor-binding element within a first loop of the guide RNA, and a second adaptor-binding element within a second loop of the guide RNA. For example, the guide RNA can be a single guide RNA comprising a CRISPR RNA (crRNA) portion fused to a transactivating CRISPR RNA (tracrRNA) portion, wherein the first loop is the tetraloop corresponding to residues 13-16 of SEQ ID NO: 11, 13, 15, or 16, and the second loop is the stem loop 2 corresponding to residues 53-56 of SEQ ID NO: 11, 13, 15, or 16. In a specific example, the adaptor-binding element comprises the sequence set forth in SEQ ID NO: 19 or 20. For example, the guide RNA can comprise the sequence set forth in any of SEQ ID NOS: 21-26. Guide RNAs are described in more detail elsewhere herein.

The fusion protein or CtIP protein in the composition or combination or methods can be in any form, such as in the form of a protein, in the form of an RNA encoding the protein, or in the form of a DNA encoding the protein (e.g., in a vector such as a recombinant adeno-associated virus (AAV) vector as described in more detail elsewhere herein). Likewise, the fusion protein or CtIP protein or the nucleic acid encoding the protein can be in any form for delivery, such as in a lipid nanoparticle as described in more detail elsewhere herein. In a specific example, the adaptor protein in the fusion protein comprises an MS2 coat protein or a functional fragment or variant thereof. For example, the adaptor protein can comprise a sequence at least 90%, 95%, 96%, 97%, 98%, 99%, or 100% identical to the sequence set forth in SEQ ID NO: 32, or can be encoded by a sequence at least 90%, 95%, 96%, 97%, 98%, 99%, or 100% identical to the sequence set forth in SEQ ID NO: 33. In one example, the CtIP protein is a human CtIP protein. In one example, the CtIP protein comprises a sequence at least 90%, 95%, 96%, 97%, 98%, 99%, or 100% identical to the sequence set forth in SEQ ID NO: 34 or is encoded by a sequence at least 90%, 95%, 96%, 97%, 98%, 99%, or 100% identical to the sequence set forth in SEQ ID NO: 36. In a specific example, the fusion protein comprises a sequence at least 90%, 95%, 96%, 97%, 98%, 99%, or 100% identical to the sequence set forth in SEQ ID NO: 30 or is encoded by a sequence at least 90%, 95%, 96%, 97%, 98%, 99%, or 100% identical to the sequence set forth in SEQ ID NO: 31. CtIP proteins, adaptor proteins, and fusion proteins are described in more detail elsewhere herein.

The i53 protein in the composition or combination or methods can be in any form, such as in the form of a protein, in the form of an RNA encoding the protein, or in the form of a DNA encoding the protein (e.g., in a vector such as a recombinant adeno-associated virus (AAV) vector as described in more detail elsewhere herein). Likewise, the i53 protein or the nucleic acid encoding the protein can be in any form for delivery, such as in a lipid nanoparticle as described in more detail elsewhere herein. In a specific example, the i53 protein comprises a sequence at least 90%, 95%, 96%, 97%, 98%, 99%, or 100% identical to the sequence set forth in SEQ ID NO: 40 or 42 or is encoded by a sequence at least 90%, 95%, 96%, 97%, 98%, 99%, or 100% identical to the sequence set forth in SEQ ID NO: 41 or 43. i53 proteins are described in more detail elsewhere herein.

The exogenous donor nucleic acid in the composition or combination or methods can be any suitable exogenous donor nucleic acid. In one example, the exogenous donor nucleic acid comprises the insert nucleic acid. The exogenous donor nucleic acid can be in a vector, such as a recombinant AAV vector. The exogenous donor nucleic acid can be any size nucleic acid. In some cases, it can be a large targeting vector (LTVEC). For example, it can be at least 10 kb in size or from about 50 kb to about 300 kb in size, or the sum total of the 5′ and 3′ homology arms can be at least 10 kb or can be from about 10 kb to about 200 kb.

If a lipid nanoparticle is used, in one example the Cas protein or nucleic acid encoding the Cas protein, the fusion protein or CtIP protein or nucleic acid encoding the fusion protein or CtIP protein, and the i53 protein or nucleic acid encoding the i53 protein can be in the same lipid nanoparticle. Alternatively, the Cas protein or nucleic acid encoding the Cas protein, the fusion protein or CtIP protein or nucleic acid encoding the fusion protein or CtIP protein, the i53 protein or nucleic acid encoding the i53 protein, and the guide RNA or DNA encoding the guide RNA can be in the same lipid nanoparticle. If a vector is used, in one example, the DNA encoding the guide RNA and the exogenous donor nucleic acid can be in the vector. Alternatively, just the exogenous donor nucleic acid can be in the vector.

In one specific example, the composition or combination comprises an RNA encoding the fusion protein or CtIP protein and an RNA encoding the i53 protein. In another specific example, the composition or combination comprises an RNA encoding the fusion protein or CtIP protein and an RNA encoding the i53 protein, wherein the RNA encoding the fusion protein and the RNA encoding the i53 protein are in a lipid nanoparticle. In another specific example, the composition or combination comprises an RNA encoding the fusion protein or CtIP protein and an RNA encoding the i53 protein, wherein the RNA encoding the fusion protein and the RNA encoding the i53 protein are in a lipid nanoparticle, and the exogenous donor nucleic acid is in a vector (e.g., a recombinant AAV vector).

In one specific example, the composition or combination comprises an RNA encoding the Cas protein, an RNA encoding the fusion protein or CtIP protein, an RNA encoding the i53 protein, and one or more DNAs encoding the guide RNA, wherein the RNA encoding the Cas protein, the RNA encoding the fusion protein, and the RNA encoding the i53 protein are in a lipid nanoparticle, and the one or more DNAs encoding the guide RNA and the exogenous donor nucleic acid are in a vector (e.g., a recombinant AAV vector).

In another specific example, the composition or combination comprises an RNA encoding the Cas protein, an RNA encoding the fusion protein or CtIP protein, an RNA encoding the i53 protein, and the guide RNA in the form of RNA, wherein the RNA encoding the Cas protein, the RNA encoding the fusion protein, the RNA encoding the i53 protein, and the guide RNA are in a lipid nanoparticle, and the exogenous donor nucleic acid is in a vector (e.g., a recombinant AAV vector).

In one specific example, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises an RNA encoding the fusion protein or CtIP protein and an RNA encoding the i53 protein. In another specific example, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises an RNA encoding the fusion protein or CtIP protein and an RNA encoding the i53 protein, and the RNA encoding the fusion protein and the RNA encoding the i53 protein are in a lipid nanoparticle. In another specific example, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises an RNA encoding the fusion protein or CtIP protein and an RNA encoding the i53 protein, and the RNA encoding the fusion protein and the RNA encoding the i53 protein are in a lipid nanoparticle, and the exogenous donor nucleic acid is in a vector (e.g., a recombinant AAV vector).

In one specific example, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises an RNA encoding the Cas protein, the composition or combination comprises an RNA encoding the fusion protein or CtIP protein, the composition or combination comprises an RNA encoding the i53 protein, the RNA encoding the Cas protein, the RNA encoding the fusion protein, and the RNA encoding the i53 protein are in a lipid nanoparticle, the composition or combination comprises the one or more DNAs encoding the guide RNA, and the one or more DNAs encoding the guide RNA and the exogenous donor nucleic acid are in a vector (e.g., a recombinant AAV vector).

In another specific example, the guide RNA comprises two adaptor-binding elements to which the adaptor protein can specifically bind, wherein a first adaptor-binding element is within a first loop of the guide RNA, and a second adaptor-binding element is within a second loop of the guide RNA, the adaptor protein comprises an MS2 coat protein or a functional fragment or variant thereof, the composition or combination comprises an RNA encoding the Cas protein, the composition or combination comprises an RNA encoding the fusion protein or CtIP protein, the composition or combination comprises an RNA encoding the i53 protein, the composition or combination comprises the guide RNA is in the form of RNA, the RNA encoding the Cas protein, the RNA encoding the fusion protein, the RNA encoding the i53 protein, and the guide RNA are in a lipid nanoparticle, and the exogenous donor nucleic acid is in a vector (e.g., a recombinant AAV vector).

The cell in the methods can be any suitable cell. Example of different cells are disclosed in more detail elsewhere herein. For example, the cell can be a mammalian cell, a rodent cell, a mouse cell, a rat cell, or a human cell. In some methods, the cells are non-cycling cells (i.e., non-dividing). In some methods, the cells are cycling (i.e., dividing) cells.

The nucleic acids or proteins used in the methods disclosed herein can be introduced into the cell by any suitable means. Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes.

In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct. In certain LNPs, the cargo can include an mRNA encoding a Cas nuclease, such as Cas9, a guide RNA or a nucleic acid encoding a guide RNA, and a nucleic acid construct. LNPs for use in the methods are described in more detail elsewhere herein.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or nucleic acid construct encoding a polypeptide of interest). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The methods can further comprise identifying a cell having a modified target genomic locus. Various methods can be used to identify cells and animals having a targeted genetic modification, such as PCR. The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

In some methods, the percentage of cells with the targeted genetic modification resulting from homology-directed repair is higher than in a control method in which CtIP protein (or CtIP fusion protein) is administered (in any form, such as protein, RNA encoding, or DNA encoding) but i53 protein is not administered (in any form). In some methods, the percentage of cells with the targeted genetic modification resulting from homology-directed repair is higher than in a control method in which i53 is administered (in any form) but CtIP protein (or CtIP fusion protein) is not administered (in any form). In some methods, the percentage of cells with the targeted genetic modification resulting from homology-directed repair is higher than in a control method in which CtIP protein (or CtIP fusion protein) and i53 protein are not administered (in any form). In some methods, the fold change in the percentage of cells with the targeted genetic modification resulting from homology-directed repair over a control method in which CtIP protein (or CtIP fusion protein) and i53 protein are not administered (in any form) is at least about 2-fold, at least about 3-fold, at least about 4-fold, at least about 5-fold, at least about 10-fold, at least about 15-fold, or at least about 20-fold. In some methods, the fold change in the percentage of cells with the targeted genetic modification resulting from homology-directed repair over a control method in which CtIP protein (or CtIP fusion protein) and i53 protein are not administered (in any form) is about 15-fold to about 25-fold, about 16-fold to about 24-fold, about 17-fold to about 23-fold, about 18-fold to about 22-fold, about 19-fold to about 21-fold, about 15-fold to about 20-fold, about 16-fold to about 20-fold, about 17-fold to about 20-fold, about 18-fold to about 20-fold, about 19-fold to about 20-fold, about 20-fold to about 25-fold, about 20-fold to about 24-fold, about 20-fold to about 23-fold, about 20-fold to about 22-fold, about 20-fold to about 21-fold, or about 20-fold.

### A. CRISPR/Cas Systems

The methods and compositions disclosed herein can utilize Clustered Regularly Interspersed Short Palindromic Repeats (CRISPR)/CRISPR-associated (Cas) systems or components of such systems to modify a genome within a cell. CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting a target locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target locus. Such CRISPR/Cas systems targeting a target locus can further comprise one or more exogenous donor sequences (e.g., targeting vectors) that target the target locus.

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (1) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5c (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, and WO 2019/067910, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. As one example, a Cas9 protein can comprise, consist essentially of, or consist of the sequence set forth in SEQ ID NO: 1. Such a Cas9 protein can be encoded by a DNA comprising, consisting essentially of, or consisting of SEQ ID NO: 2.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12c) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) and a substitution at position D10 (e.g., D10A substitution) to generate a nuclease-inactive Cas protein. See, e.g., WO 2016/106236, herein incorporated by reference in its entirety for all purposes. Examples of inactivating mutations in the catalytic domains of Nme2Cas9 are also known (e.g., combination of D16A and H588A). Examples of inactivating mutations in the catalytic domains of St1Cas9 are also known (e.g., combination of D9A, D598A, H599A, and N622A). Examples of inactivating mutations in the catalytic domains of St3Cas9 are also known (e.g., combination of D10A and N870A). Examples of inactivating mutations in the catalytic domains of CjCas9 are also known (e.g., combination of D8A and H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 3) or PKKKRRV (SEQ ID NO: 4). The NLS may be a bipartite sequence, such as the NLS of nucleoplasmin, KRPAATKKAGQAKKKK (SEQ ID NO: 5). In a specific example, a single PKKKRKV (SEQ ID NO: 3) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for case of tracking or purification, such as a fluorescent protein, a purification tag, or an epitope tag. Examples of fluorescent proteins include green fluorescent proteins (e.g., GFP, GFP-2, tagGFP, turboGFP, cGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, Zs Yellow1), blue fluorescent proteins (e.g., cBFP, cBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, cqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, a Cas9 mRNA and a gRNA targeting a target genomic locus can be delivered via LNP-mediated delivery, or a DNA encoding Cas9 and a DNA encoding a gRNA targeting a target genomic locus can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl pseudouridine can be used. As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). Likewise, Cas mRNAs can be modified by depletion of uridine using synonymous codons. For example, capped and polyadenylated Cas mRNA fully substituted with pseudouridine can be used.

Cas mRNAs can comprise a modified uridine at least at one, a plurality of, or all uridine positions. The modified uridine can be a uridine modified at the 5′ position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11): E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its DI subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

Any of the above modifications to Cas mRNAs (e.g., for improved stability and/or immunogenicity properties) can also be used for RNAs encoding the CtIP fusion proteins or i53 proteins disclosed herein.

### (2) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called a “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a guide RNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 6) or GUUUUAGAGCUAUGCUGUUUUG (SEQ ID NO: 7). Any DNA-targeting segment can be joined to the 5′ end of SEQ ID NO: 6 or SEQ ID NO: 7 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences comprise, consist essentially of, or consist of any one of

(SEQ ID NO: 8)
AGCAUAGCAAGUUAAAAUAAGGCUAGUCCGUUAUCAACUUGAAAAA
GUGGCACCGAGUCGGUGCUUU,
(SEQ ID NO: 9)
AAACAGCAUAGCAAGUUAAAAUAAGGCUAGUCCGUUAUCAACUUGA
AAAAGUGGCACCGAGUCGGUGCUUUU,
or
(SEQ ID NO: 10)
GUUGGAACCAUUCAAAACAGCAUAGCAAGUUAAAAUAAGGCUAGUC
CGUUAUCAACUUGAAAAAGUGGCACCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, about 18, about 19, or about 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In Ir one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, about or more than about 26, about or more than about 32, about or more than about 45, about or more than about 48, about or more than about 54, about or more than about 63, about or more than about 67, about or more than about 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 11); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 12); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 13); GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 14); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 15); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 16); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 17); or GUUUUAGAGCUAGAAAUAGCAAGUUAAAAUAAGGCUAGUCCGUUAUCAACUUGG CACCGAGUCGGUGC (version 8; SEQ ID NO: 18). In some guide sgRNAs, the four terminal U residues of version 6 are not present. In some sgRNAs, only 1, 2, or 3 of the four terminal U residues of version 6 are present. Guide RNAs targeting can include, for example, any DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). Guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, such as proteins that affect homology-directed repair processes); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification)); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such as those listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively, or additionally, a modified gRNA can comprise at least one modified residue at or near the 5′ end. Alternatively, or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

In some guide RNAs (e.g., single guide RNAs), at least one loop (e.g., two loops) of the guide RNA is modified by insertion of a distinct RNA sequence that binds to one or more adaptors (i.e., adaptor proteins or domains). Such adaptor proteins can be used to further recruit one or more heterologous functional domains, such as proteins that affect homology-directed repair processes in a cell. Examples of fusion proteins comprising such adaptor proteins (i.e., fusion proteins) are disclosed elsewhere herein. For example, an MS2-binding loop ggccAACAUGAGGAUCACCCAUGUCUGCAGggcc (SEQ ID NO: 19) may replace nucleotides +13 to +16 and nucleotides +53 to +56 of the sgRNA scaffold (backbone) set forth in SEQ ID NO: 11, 13, 15, or 16 or the sgRNA backbone for the S. pyogenes CRISPR/Cas9 system described in WO 2016/049258 and Konermann et al. (2015) Nature 517(7536):583-588, each of which is herein incorporated by reference in its entirety for all purposes. The guide RNA numbering used herein refers to the nucleotide numbering in the guide RNA scaffold sequence (i.e., the sequence downstream of the DNA-targeting segment of the guide RNA). For example, the first nucleotide of the guide RNA scaffold is +1, the second nucleotide of the scaffold is +2, and so forth. Residues corresponding with nucleotides +13 to +16 in SEQ ID NO: 11, 13, 15, or 16 are the loop sequence in the region spanning nucleotides +9 to +21 in SEQ ID NO: 11, 13, 15, or 16, a region referred to herein as the tetraloop. Residues corresponding with nucleotides +53 to +56 in SEQ ID NO: 11, 13, 15, or 16 are the loop sequence in the region spanning nucleotides +48 to +61 in SEQ ID NO: 11, 13, 15, or 16, a region referred to herein as the stem loop 2. Other stem loop sequences in SEQ ID NO: 11, 13, 15, or 16 comprise stem loop 1 (nucleotides +33 to +41) and stem loop 3 (nucleotides +63 to +75). The resulting structure is an sgRNA scaffold in which each of the tetraloop and stem loop 2 sequences have been replaced by an MS2 binding loop. The tetraloop and stem loop 2 protrude from the Cas9 protein in such a way that adding an MS2-binding loop should not interfere with any Cas9 residues. Additionally, the proximity of the tetraloop and stem loop 2 sites to the DNA indicates that localization to these locations could result in a high degree of interaction between the DNA and any recruited protein, such as a protein that affects homology-directed repair processes. Thus, in some sgRNAs, nucleotides corresponding to +13 to +16 and/or nucleotides corresponding to +53 to +56 of the guide RNA scaffold set forth in SEQ ID NO: 11, 13, 15, or 16 or corresponding residues when optimally aligned with any of these scaffold/backbones are replaced by the distinct RNA sequences capable of binding to one or more adaptor proteins or domains. Alternatively, or additionally, adaptor-binding sequences can be added to the 5′ end or the 3′ end of a guide RNA. An exemplary guide RNA scaffold comprising MS2-binding loops in the tetraloop and stem loop 2 regions can comprise, consist essentially of, or consist of the sequence set forth in SEQ ID NO: 21, 22, or 23 (e.g., SEQ ID NO: 23). An exemplary generic single guide RNA comprising MS2-binding loops in the tetraloop and stem loop 2 regions can comprise, consist essentially of, or consist of the sequence set forth in SEQ ID NO: 24, 25, or 26 (e.g., SEQ ID NO: 26).

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

### (3) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Target genes can include genes expressed in particular organs or tissues, such as the liver. Target genes can include disease-associated genes. A disease-associated gene refers to any gene that yields transcription or translation products at an abnormal level or in an abnormal form in cells derived from a disease-affected tissues compared with tissues or cells of a non-disease control. It may be a gene that becomes expressed at an abnormally high level, where the altered expression correlates with the occurrence and/or progression of the disease. A disease-associated gene also refers to a gene possessing a mutation or genetic variation that is responsible for the etiology of a disease. The transcribed or translated products may be known or unknown, and may be at a normal or abnormal level. Target genes can also be genes involved in pathways related to a disease or condition or genes that when overexpressed can model such diseases or conditions. Target genes can also be genes expressed or overexpressed in one or more types of cancer. See, e.g., Santarius et al. (2010) Nat. Rev. Cancer 10(1):59-64, herein incorporated by reference in its entirety for all purposes.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG or N₂₀NGG. See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of the above sequences, including the 5′ G or GG and the 3′ GG or NGG. Yet other guide RNA target sequences plus PAMs can have between 14 and 20 nucleotides in length of the above sequences.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, at least 3, at least 4, at least 5, at least 6, at least 7, at least 8, at least 9, at least 10, at least 15, at least 20, at least 25, at least 30, at least 40, at least 50, at least 75, at least 100, at least 250, at least 500, or at least 1,000 base pairs.

### B. CtBP-Interacting Protein (CtIP) Fusion Proteins

The compositions or combinations and corresponding methods disclosed herein make use of CtIP proteins that can be part of a fusion protein that can bind to the guide RNAs disclosed elsewhere herein. The fusion proteins disclosed herein are useful in the methods described herein to bring the CtIP near the cleaved target genomic locus to promote homology-directed repair. Nucleic acids encoding the fusion proteins can be genomically integrated in a cell or animal (e.g., a cell or animal comprising a genomically integrated fusion protein expression cassette), or the fusion proteins or nucleic acids can be introduced into such cells and animals using methods disclosed elsewhere herein (e.g., LNP-mediated delivery or AAV-mediated delivery).

Such fusion comprise: (a) an adaptor (i.e., adaptor domain or adaptor protein) that specifically binds to an adaptor-binding element within a guide RNA; and (b) a CtIP protein. For example, the fusion protein can comprise: (a) an MS2 coat protein adaptor that specifically binds to one or more MS2 aptamers in a guide RNA (e.g., two MS2 aptamers in separate locations in a guide RNA); and (b) a CtIP protein.

The CtIP can be fused directly to the adaptor. Alternatively, the CtIP can be linked to the adaptor via a linker or a combination of linkers or via one or more additional domains. Linkers that can be used in these fusion proteins can include any sequence that does not interfere with the function of the fusion proteins. Exemplary linkers are short (e.g., 2-20 amino acids) and are typically flexible (e.g., comprising amino acids with a high degree of freedom such as glycine, alanine, and serine). Some specific examples of linkers comprise one or more units consisting of GGGS (SEQ ID NO: 28) or GGGGS (SEQ ID NO: 29), such as two, three, four, or more repeats of GGGS (SEQ ID NO: 28) or GGGGS (SEQ ID NO: 29) in any combination. Other linker sequences can also be used.

The CtIP and the adaptor can be in any order within the fusion protein. As one option, the CtIP can be C-terminal to the adaptor and the adaptor can be N-terminal to the CtIP. For example, the CtIP can be at the C-terminus of the fusion protein, and the adaptor can be at the N-terminus of the fusion protein. However, the CtIP can be C-terminal to the adaptor without being at the C-terminus of the fusion protein (e.g., if a nuclear localization signal is at the C-terminus of the fusion protein). Likewise, the adaptor can be N-terminal to the CtIP without being at the N-terminus of the fusion protein (e.g., if a nuclear localization signal is at the N-terminus of the fusion protein). As another option, the CtIP can be N-terminal to the adaptor and the adaptor can be C-terminal to the CtIP. For example, the CtIP can be at the N-terminus of the fusion protein, and the adaptor can be at the C-terminus of the fusion protein.

The fusion proteins described herein can also be operably linked or fused to additional heterologous polypeptides. The fused or linked heterologous polypeptide can be located at the N-terminus, the C-terminus, or anywhere internally within the fusion protein. For example, a CtIP protein can further comprise a nuclear localization signal. A specific example of such a protein comprises an MS2 coat protein (adaptor) linked (either directly or via an NLS) to a CtIP protein C-terminal to the MS2 coat protein (MCP). Such a protein can comprise from N-terminus to C-terminus: an MCP; a nuclear localization signal; and a CtIP protein. In one example, a fusion protein can comprise an amino acid sequence at least 85%, at least 90%, at least 91%, at least 92%, at least 93%, at least 94%, at least 95%, at least 96%, at least 97%, at least 98%, at least 99%, or 100% identical to the fusion protein sequence set forth in SEQ ID NO: 30. In another example, a fusion protein can consist essentially of an amino acid sequence at least 85%, at least 90%, at least 91%, at least 92%, at least 93%, at least 94%, at least 95%, at least 96%, at least 97%, at least 98%, at least 99%, or 100% identical to the fusion protein sequence set forth in SEQ ID NO: 30. In another example, a fusion protein can consist of an amino acid sequence at least 85%, at least 90%, at least 91%, at least 92%, at least 93%, at least 94%, at least 95%, at least 96%, at least 97%, at least 98%, at least 99%, or 100% identical to the fusion protein sequence set forth in SEQ ID NO: 30. In one example, a fusion protein can be encoded by a nucleic acid comprising a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 31. In another example, a fusion protein can be encoded by a nucleic acid consisting essentially of a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 31. In another example, a fusion protein can be encoded by a nucleic acid consisting of a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 31.

In one example, the fusion protein comprises the sequence set forth in SEQ ID NO: 30. In another example, the fusion protein consists essentially of the sequence set forth in SEQ ID NO: 30. In another example, the fusion protein consists of the sequence set forth in SEQ ID NO: 30. In one example, a nucleic acid encoding the fusion protein comprises the sequence set forth in SEQ ID NO: 31. In another example, a nucleic acid encoding the fusion protein consists essentially of the sequence set forth in SEQ ID NO: 31. In another example, a nucleic acid encoding the fusion protein consists of the sequence set forth in SEQ ID NO: 31.

Fusion proteins can also be fused or linked to one or more heterologous polypeptides that provide for subcellular localization. Such heterologous polypeptides can include, for example, one or more nuclear localization signals (NLS) such as the SV40 NLS and/or an alpha-importin NLS for targeting to the nucleus, a mitochondrial localization signal for targeting to the mitochondria, an ER retention signal, and the like. See, e.g., Lange et al. (2007) J. Biol. Chem. 282:5101-5105, herein incorporated by reference in its entirety for all purposes. An NLS can comprise, for example, a stretch of basic amino acids, and can be a monopartite sequence or a bipartite sequence. Optionally, the fusion protein comprises two or more NLSs, including an NLS (e.g., an alpha-importin NLS) at the N-terminus and/or an NLS (e.g., an SV40 NLS) at the C-terminus.

Fusion proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO2013/176772, each of which is herein incorporated by reference in its entirety for all purposes. As another example, fusion proteins can be fused or linked to a heterologous polypeptide providing increased or decreased stability.

Fusion proteins can also be operably linked to a heterologous polypeptide for case of tracking or purification, such as a fluorescent protein, a purification tag, or an epitope tag. Examples of fluorescent proteins include green fluorescent proteins (e.g., GFP, GFP-2, tagGFP, turboGFP, cGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, Zs Yellow1), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, cqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Fusion proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the fusion protein. Likewise, the fusion protein can be tethered to the 5′ end, the 3′ end, or to an internal region within the labeled nucleic acid. That is, the labeled nucleic acid can be tethered in any orientation and polarity.

### (1) Adaptor Proteins or Adaptor Domains

Adaptors (i.e., adaptor domains or adaptor proteins) are nucleic-acid-binding domains (e.g., DNA-binding domains and/or RNA-binding domains) that specifically recognize and bind to distinct sequences (e.g., bind to distinct DNA and/or RNA sequences such as aptamers in a sequence-specific manner). Aptamers include nucleic acids that, through their ability to adopt a specific three-dimensional conformation, can bind to a target molecule with high affinity and specificity. Such adaptors can bind, for example, to a specific RNA sequence and secondary structure. These sequences (i.e., adaptor-binding elements) can be engineered into a guide RNA. For example, an MS2 aptamer can be engineered into a guide RNA to specifically bind an MS2 coat protein (MCP). In one example, the adaptor can comprise an amino acid sequence at least 85%, at least 90%, at least 91%, at least 92%, at least 93%, at least 94%, at least 95%, at least 96%, at least 97%, at least 98%, at least 99%, or 100% identical to the MCP sequence set forth in SEQ ID NO: 32. In another example, the adaptor can consist essentially of an amino acid sequence at least 85%, at least 90%, at least 91%, at least 92%, at least 93%, at least 94%, at least 95%, at least 96%, at least 97%, at least 98%, at least 99%, or 100% identical to the MCP sequence set forth in SEQ ID NO: 32. In another example, the adaptor can consist of an amino acid sequence at least 85%, at least 90%, at least 91%, at least 92%, at least 93%, at least 94%, at least 95%, at least 96%, at least 97%, at least 98%, at least 99%, or 100% identical to the MCP sequence set forth in SEQ ID NO: 32. In one example, the adaptor can be encoded by a nucleic acid comprising a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 33. In another example, the adaptor can be encoded by a nucleic acid consisting essentially of a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 33. In another example, the adaptor can be encoded by a nucleic acid consisting of a sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 33.

In one example, the adaptor comprises the sequence set forth in SEQ ID NO: 32. In another example, the adaptor consists essentially of the sequence set forth in SEQ ID NO: 32. In another example, the adaptor consists of the sequence set forth in SEQ ID NO: 32. In one example, a nucleic acid encoding the adaptor comprises the sequence set forth in SEQ ID NO: 33. In another example, a nucleic acid encoding the adaptor consists essentially of the sequence set forth in SEQ ID NO: 33. In another example, a nucleic acid encoding the adaptor consists of the sequence set forth in SEQ ID NO: 33.

Some specific examples of adaptors and targets include RNA-binding protein/aptamer combinations that exist within the diversity of bacteriophage coat proteins. For example, the following adaptor proteins or functional fragments or variants thereof can be used: MS2 coat protein (MCP), PP7, Qβ, F2, GA, fr, JP501, M12, R17, BZ13, JP34, JP500, KU1, M11, MX1, TW18, VK, SP, FI, ID2, NL95, TW19, AP205, φCb5, Φ Cb8r, Φ Cb12r, ΦCb23r, 7s, and PRR1. See, e.g., WO 2016/049258, herein incorporated by reference in its entirety for all purposes. A functional fragment or functional variant of an adaptor protein is one that retains the ability to bind to a specific adaptor-binding element (e.g., ability to bind to a specific adaptor-binding sequence in a sequence-specific manner). For example, a PP7 Pseudomonas bacteriophage coat protein variant can be used in which amino acids 68-69 are mutated to SG and amino acids 70-75 are deleted from the wild type protein. See, e.g., Wu et al. (2012) Biophys. J. 102(12):2936-2944 and Chao et al. (2007) Nat. Struct. Mol. Biol. 15(1):103-105, each of which is herein incorporated by reference in its entirety for all purposes. Likewise, an MCP variant may be used, such as a N55K mutant. See, e.g., Spingola and Peabody (1994) J. Biol. Chem. 269(12):9006-9010, herein incorporated by reference in its entirety for all purposes.

Other examples of adaptor proteins that can be used include all or part of (e.g., the DNA-binding from) endoribonuclease Csy4 or the lambda N protein. See, e.g., U S 2016/0312198, herein incorporated by reference in its entirety for all purposes.

### (2) CtIP

The compositions or combinations and corresponding methods disclosed herein make use of CtIP proteins. In one example, the CtIP protein used in the methods, compositions, and combinations disclosed herein is a human CtIP protein. Human C-terminal binding protein (CtBP)-interacting protein (CtIP) (also called DNA endonuclease RBBP8, RBBP8, retinoblastoma-binding protein 8, RBBP-8, retinoblastoma-interacting protein and myosin-like, RIM, sporulation in the absence of SPO11 protein 2 homolog, SAE2) is assigned UniProt Reference No. Q99807 and is encoded by the gene CTIP (also called RBBP8), which is assigned NCBI GeneID 5932. The gene is at location 18q11.2 on chromosome 18 (Assembly: GRCh38.p14 (GCF_000001405.40); Location: NC_000018.10 (22914139 . . . 23026486)). The canonical isoform of human CtIP (UniProt Reference Q99708-1, NCBI Reference No. NP_002885.1) is set forth in SEQ ID NO: 37. An mRNA encoding the canonical isoform is assigned NCBI Reference No. NM_002894.3 (SEQ ID NO: 38). A coding sequence for the canonical isoform is assigned CCDS No. CCDS11875.1 (SEQ ID NO: 39). Another isoform of human CtIP (NCBI Reference No. AAC14371.1) is set forth in SEQ ID NO: 34. An mRNA encoding the canonical isoform is assigned NCBI Reference No. U72066.1 (SEQ ID NO: 35). A coding sequence for the canonical isoform is set forth in SEQ ID NO: 36. CtIP is an endonuclease that cooperates with the MRE11-RAD50-NBN (MRN) complex in DNA-end resection, the first step of double-strand break repair through the homologous recombination pathway.

In one example, the CtIP protein used in the methods, compositions, and combinations disclosed herein comprises a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 34. In another example, the CtIP protein used in the methods, compositions, and combinations disclosed herein consists essentially of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 34. In another example, the CtIP protein used in the methods, compositions, and combinations disclosed herein consists of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 34. In another example, the CtIP protein used in the methods, compositions, and combinations disclosed herein comprises the sequence set forth in SEQ ID NO: 34. In another example, the CtIP protein used in the methods, compositions, and combinations disclosed herein consists essentially of the sequence set forth in SEQ ID NO: 34. In another example, the CtIP protein used in the methods, compositions, and combinations disclosed herein consists of the sequence set forth in SEQ ID NO: 34.

In one example, the CtIP protein is encoded by a nucleic acid that comprises a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 36. In another example, the CLIP protein is encoded by a nucleic acid that consists essentially of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 36. In another example, the CtIP protein is encoded by a nucleic acid that consists of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 36. In another example, the CtIP protein is encoded by a nucleic acid that comprises the sequence set forth in SEQ ID NO: 36. In another example, the CtIP protein is encoded by a nucleic acid that consists essentially of the sequence set forth in SEQ ID NO: 36. In another example, the CtIP protein is encoded by a nucleic acid that consists of the sequence set forth in SEQ ID NO: 36.

### C. Inhibitor of 53BP1

The compositions or combinations and corresponding methods disclosed herein make use of inhibitor of 53BP1 (153) proteins. i53 is a variant of ubiquitin that blocks accumulation of 53BP1 at sites of DNA damage. i53 binds and occludes the ligand binding site of the 53BP1 tumor domain, blocking its ability to accumulate at sites of DNA damage.

In one example, the i53 protein used in the methods, compositions, and combinations disclosed herein comprises a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 40 or 42 (e.g., 40). In another example, the i53 protein used in the methods, compositions, and combinations disclosed herein consists essentially of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 40 or 42 (e.g., 40). In another example, the i53 protein used in the methods, compositions, and combinations disclosed herein consists of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 40 or 42 (e.g., 40). In another example, the i53 protein used in the methods, compositions, and combinations disclosed herein comprises the sequence set forth in SEQ ID NO: 40 or 42 (e.g., 40). In another example, the i53 protein used in the methods, compositions, and combinations disclosed herein consists essentially of the sequence set forth in SEQ ID NO: 40 or 42 (e.g., 40). In another example, the i53 protein used in the methods, compositions, and combinations disclosed herein consists of the sequence set forth in SEQ ID NO: 40 or 42 (e.g., 40).

In one example, the i53 protein is encoded by a nucleic acid that comprises a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 41 or 43 (e.g., 41). In another example, the i53 protein is encoded by a nucleic acid that consists essentially of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 41 or 43 (e.g., 41). In another example, the i53 protein is encoded by a nucleic acid that consists of a sequence at least about 90%, at least about 91%, at least about 92%, at least about 93%, at least about 94%, at least about 95%, at least about 96%, at least about 97%, at least about 98%, at least about 99%, or about 100% identical to SEQ ID NO: 41 or 43 (e.g., 41). In another example, the i53 protein is encoded by a nucleic acid that comprises the sequence set forth in SEQ ID NO: 41 or 43 (e.g., 41). In another example, the i53 protein used in the methods, compositions, and combinations disclosed herein consists essentially of the sequence set forth in SEQ ID NO: 41 or 43 (e.g., 41). In another example, the i53 protein is encoded by a nucleic acid that consists of the sequence set forth in SEQ ID NO: 41 or 43 (e.g., 41).

### D. Exogenous Donor Nucleic Acids

The methods and compositions disclosed herein utilize exogenous donor nucleic acids to modify the target genomic locus following cleavage with a Cas protein. In such methods, the Cas protein cleaves the target genomic locus (e.g., to create a double-strand break), and the exogenous donor nucleic acid recombines the target nucleic acid through a homology-directed repair event. Optionally, repair with the exogenous donor nucleic acid removes or disrupts the guide RNA target sequence or the Cas cleavage site so that alleles that have been targeted cannot be re-targeted by the Cas protein. In some cases, an exogenous repair template is flanked by guide RNA target sequences that are cleaved by the Cas protein within the cell.

Exogenous donor nucleic acids can comprise deoxyribonucleic acid (DNA) or ribonucleic acid (RNA), they can be single-stranded or double-stranded, and they can be in linear or circular form. For example, an exogenous donor nucleic acid can be a single-stranded oligodeoxynucleotide (ssODN). See, e.g., Yoshimi et al. (2016) Nat. Commun. 7:10431, herein incorporated by reference in its entirety for all purposes. An exemplary exogenous donor nucleic acid is between about 50 nucleotides to about 5 kb in length, is between about 50 nucleotides to about 3 kb in length, or is between about 50 to about 1,000 nucleotides in length. Other exemplary exogenous donor nucleic acids are between about 40 to about 200 nucleotides in length. For example, an exogenous donor nucleic acid can be between about 50-60, 60-70, 70-80, 80-90, 90-100, 100-110, 110-120, 120-130, 130-140, 140-150, 150-160, 160-170, 170-180, 180-190, or 190-200 nucleotides in length. Alternatively, an exogenous donor nucleic acid can be between about 50-100, 100-200, 200-300, 300-400, 400-500, 500-600, 600-700, 700-800, 800-900, or 900-1000 nucleotides in length. Alternatively, an exogenous donor nucleic acid can be between about 1-1.5, 1.5-2, 2-2.5, 2.5-3, 3-3.5, 3.5-4, 4-4.5, or 4.5-5 kb in length. Alternatively, an exogenous donor nucleic acid can be, for example, no more than 5 kb, 4.5 kb, 4 kb, 3.5 kb, 3 kb, 2.5 kb, 2 kb, 1.5 kb, 1 kb, 900 nucleotides, 800 nucleotides, 700 nucleotides, 600 nucleotides, 500 nucleotides, 400 nucleotides, 300 nucleotides, 200 nucleotides, 100 nucleotides, or 50 nucleotides in length. Exogenous donor nucleic acids (e.g., targeting vectors) can also be longer.

In one example, an exogenous donor nucleic acid is an ssODN that is between about 80 nucleotides and about 200 nucleotides in length. In another example, an exogenous donor nucleic acid is an ssODN that is between about 80 nucleotides and about 3 kb in length. Such an ssODN can have homology arms, for example, that are each between about 40 nucleotides and about 60 nucleotides in length. Such an ssODN can also have homology arms, for example, that are each between about 30 nucleotides and 100 nucleotides in length. The homology arms can be symmetrical (e.g., each 40 nucleotides or each 60 nucleotides in length), or they can be asymmetrical (e.g., one homology arm that is 36 nucleotides in length, and one homology arm that is 91 nucleotides in length).

Exogenous donor nucleic acids can include modifications or sequences that provide for additional desirable features (e.g., modified or regulated stability; tracking or detecting with a fluorescent label; a binding site for a protein or protein complex; and so forth). Exogenous donor nucleic acids can comprise one or more fluorescent labels, purification tags, epitope tags, or a combination thereof. For example, an exogenous donor nucleic acid can comprise one or more fluorescent labels (e.g., fluorescent proteins or other fluorophores or dyes), such as at least 1, at least 2, at least 3, at least 4, or at least 5 fluorescent labels. Exemplary fluorescent labels include fluorophores such as fluorescein (e.g., 6-carboxyfluorescein (6-FAM)), Texas Red, HEX, Cy3, Cy5, Cy5.5, Pacific Blue, 5-(and -6)-carboxytetramethylrhodamine (TAMRA), and Cy7. A wide range of fluorescent dyes are available commercially for labeling oligonucleotides (e.g., from Integrated DNA Technologies). Such fluorescent labels (e.g., internal fluorescent labels) can be used, for example, to detect an exogenous donor nucleic acid that has been directly integrated into a cleaved target nucleic acid having protruding ends compatible with the ends of the exogenous donor nucleic acid. The label or tag can be at the 5′ end, the 3′ end, or internally within the exogenous donor nucleic acid. For example, an exogenous donor nucleic acid can be conjugated at 5′ end with the IR 700 fluorophore from Integrated DNA Technologies (5′IRDYE® 700).

The exogenous donor nucleic acids disclosed herein comprise homology arms. If the exogenous donor nucleic acid also comprises a nucleic acid insert, the homology arms can flank the nucleic acid insert. For case of reference, the homology arms are referred to herein as 5′ and 3′ (i.e., upstream and downstream) homology arms. This terminology relates to the relative position of the homology arms to the nucleic acid insert within the exogenous donor nucleic acid. The 5′ and 3′ homology arms correspond to regions within the target genomic locus, which are referred to herein as “5′ target sequence” and “3′ target sequence,” respectively.

A homology arm and a target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. Moreover, a corresponding region of homology between the homology arm and the corresponding target sequence can be of any length that is sufficient to promote homologous recombination. Exemplary homology arms are between about 25 nucleotides to about 2.5 kb in length, are between about 25 nucleotides to about 1.5 kb in length, or are between about 25 to about 500 nucleotides in length. For example, a given homology arm (or each of the homology arms) and/or corresponding target sequence can comprise corresponding regions of homology that are between about 25-30, 30-40, 40-50, 50-60, 60-70, 70-80, 80-90, 90-100, 100-150, 150-200, 200-250, 250-300, 300-350, 350-400, 400-450, or 450-500 nucleotides in length, such that the homology arms have sufficient homology to undergo homologous recombination with the corresponding target sequences within the target nucleic acid. Alternatively, a given homology arm (or each homology arm) and/or corresponding target sequence can comprise corresponding regions of homology that are between about 0.5 kb to about 1 kb, about 1 kb to about 1.5 kb, about 1.5 kb to about 2 kb, or about 2 kb to about 2.5 kb in length. For example, the homology arms can each be about 750 nucleotides in length. The homology arms can be symmetrical (each about the same size in length), or they can be asymmetrical (one longer than the other).

When a CRISPR/Cas system is used in combination with an exogenous donor nucleic acid, the 5′ and 3′ target sequences are optionally located in sufficient proximity to the Cas cleavage site (e.g., within sufficient proximity to the guide RNA target sequence) so as to promote the occurrence of a homologous recombination event between the target sequences and the homology arms upon a single-strand break (nick) or double-strand break at the Cas cleavage site. The term “Cas cleavage site” includes a DNA sequence at which a nick or double-strand break is created by a Cas enzyme (e.g., a Cas9 protein complexed with a guide RNA). The target sequences within the targeted locus that correspond to the 5′ and 3′ homology arms of the exogenous donor nucleic acid are “located in sufficient proximity” to a Cas cleavage site if the distance is such as to promote the occurrence of a homologous recombination event between the 5′ and 3′ target sequences and the homology arms upon a single-strand break or double-strand break at the Cas cleavage site. Thus, the target sequences corresponding to the 5′ and/or 3′ homology arms of the exogenous donor nucleic acid can be, for example, within at least 1 nucleotide of a given Cas cleavage site or within at least 10 nucleotides to about 1,000 nucleotides of a given Cas cleavage site. As an example, the Cas cleavage site can be immediately adjacent to at least one or both of the target sequences.

The spatial relationship of the target sequences that correspond to the homology arms of the exogenous donor nucleic acid and the Cas cleavage site can vary. For example, target sequences can be located 5′ to the Cas cleavage site, target sequences can be located 3′ to the Cas cleavage site, or the target sequences can flank the Cas cleavage site.

Exogenous donor nucleic acids can also comprise nucleic acid inserts including segments of DNA to be integrated at target genomic loci. Integration of a nucleic acid insert at a target genomic locus can result in addition of a nucleic acid sequence of interest to the target genomic locus, deletion of a nucleic acid sequence of interest at the target genomic locus, or replacement of a nucleic acid sequence of interest at the target genomic locus (i.e., deletion and insertion). Some exogenous donor nucleic acids are designed for insertion of a nucleic acid insert at a target genomic locus without any corresponding deletion at the target genomic locus. Other exogenous donor nucleic acids are designed to delete a nucleic acid sequence of interest at a target genomic locus without any corresponding insertion of a nucleic acid insert. Yet other exogenous donor nucleic acids are designed to delete a nucleic acid sequence of interest at a target genomic locus and replace it with a nucleic acid insert.

The nucleic acid insert or the corresponding nucleic acid at the target genomic locus being deleted and/or replaced can be various lengths. An exemplary nucleic acid insert or corresponding nucleic acid at the target genomic locus being deleted and/or replaced is between about 1 nucleotide to about 5 kb in length or is between about 1 nucleotide to about 1,000 nucleotides in length. For example, a nucleic acid insert or a corresponding nucleic acid at the target genomic locus being deleted and/or replaced can be between about 1-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, or 190-120 nucleotides in length. Likewise, a nucleic acid insert or a corresponding nucleic acid at the target genomic locus being deleted and/or replaced can be between 1-100, 100-200, 200-300, 300-400, 400-500, 500-600, 600-700, 700-800, 800-900, or 900-1000 nucleotides in length. Likewise, a nucleic acid insert or a corresponding nucleic acid at the target genomic locus being deleted and/or replaced can be between about 1-1.5, 1.5-2, 2-2.5, 2.5-3, 3-3.5, 3.5-4, 4-4.5, or 4.5-5 kb in length or longer.

The nucleic acid insert can comprise a sequence that is homologous or orthologous to all or part of sequence targeted for replacement. For example, the nucleic acid insert can comprise a sequence that comprises one or more point mutations (e.g., 1, 2, 3, 4, 5, or more) compared with a sequence targeted for replacement at the target genomic locus. Optionally, such point mutations can result in a conservative amino acid substitution (e.g., substitution of aspartic acid [Asp, D] with glutamic acid [Glu, E]) in the encoded polypeptide.

In some cases, the exogenous donor nucleic acid can be a “large targeting vector” or “LTVEC,” which includes targeting vectors that comprise homology arms that correspond to and are derived from nucleic acid sequences larger than those typically used by other approaches intended to perform homologous recombination in cells. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. In one example, an LTVEC can be between about 50 kb and about 300 kb in length, or the sum total of the 5′ and 3′ homology arms can be from about 10 kb to about 200 kb in length.

### E. Vectors

The nucleic acids disclosed herein (e.g., DNA encoding a CtIP fusion protein, DNA encoding an i53 protein, DNA encoding a Cas protein, DNA encoding a guide RNA, an exogenous donor nucleic acid, or a combination thereof such as an exogenous donor nucleic acid and a DNA encoding a guide RNA) can be provided in a vector. A vector can comprise additional sequences such as, for example, replication origins, promoters, and genes encoding antibiotic resistance.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper components to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹² to about 10¹⁶ vg/mL. Other exemplary viral titers (e.g., AAV titers) include about 10¹² to about 10¹⁶ vg/kg of body weight.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes, a gene expression cassette can be placed between ITR sequences. Typically, rAAV genome cassettes comprise of a promoter to drive expression of a transgene, followed by a polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, AAV-DJ, and AAVhu.37, and particularly AAV8. In a specific example, the AAV vector can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector. In another specific example, the AAV vector can be recombinant AAV2 (rAAV2).

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

### F. Lipid Nanoparticles

The different components of the compositions or combinations disclosed herein (e.g., CtIP fusion protein or DNA or RNA encoding, i53 protein or DNA or RNA encoding, Cas protein or DNA or RNA encoding, guide RNA or DNA encoding, exogenous donor nucleic acid, or a combination thereof (such as an RNA encoding a Cas protein, an RNA encoding a CtIP fusion protein, an RNA encoding an i53 protein, and optionally a guide RNA) can be provided in a lipid nanoparticle.

Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

In a specific example, an RNA encoding a CLIP fusion protein and an RNA encoding an i53 protein are each introduced via LNP-mediated delivery in the same LNP. In another specific example, an RNA encoding a Cas protein, an RNA encoding a CtIP fusion protein, and an RNA encoding an i53 protein are each introduced via LNP-mediated delivery in the same LNP. In another specific example, an RNA encoding a Cas protein, an RNA encoding a CLIP fusion protein, an RNA encoding an i53 protein, and optionally a guide RNA are each introduced via LNP-mediated delivery in the same LNP. As discussed in more detail elsewhere herein, one or more of the RNAs can be modified. Delivery through such methods can result in transient Cas, CtIP fusion protein, or i53 protein expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. See, e.g., WO 2016/010840 A1 and WO 2017/173054 A1, each of which is herein incorporated by reference in its entirety for all purposes. An exemplary lipid nanoparticle can comprise a cationic lipid and one or more other components.

In some LNPs, the cargo can comprise Cas mRNA (e.g., Cas9 mRNA) and gRNA. The Cas mRNA and gRNAs can be in different ratios. In some LNPs, the cargo can comprise a nucleic acid construct encoding a product of interest (e.g., polypeptide of interest) and gRNA. The nucleic acid construct encoding a product of interest (e.g., polypeptide of interest) and gRNAs can be in different ratios.

Examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. A specific example of using LNPs to deliver to the brain is disclosed in Nabhan et al. (2016) Sci. Rep. 6:20019, herein incorporated by reference in its entirety for all purposes.

### G. Cells and Animals

The cells targeted in the methods disclosed herein can be, for example, mammalian, non-human mammalian, or human. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). The term “non-human” excludes humans. In one specific example, the cells are mammalian cells. In another example, the cells are rodent cells. In another example, the cells are mouse or rat cells. In another example, the cells are mouse cells. In another example, the cells are rat cells. In another example, the cells are human cells. In one example, the cells are non-cycling cells (i.e., non-dividing). In another example, the cells are cycling (i.e., dividing) cells.

The cells can be isolated cells (e.g., in vitro) or can be in vivo within a subject (e.g., animal or mammal). Cells can also be any type of undifferentiated or differentiated state. In one example, the cells are liver cells. The cells provided herein can be normal, healthy cells, or can be diseased cells.

In some embodiments, the cells can be induced pluripotent stem cells (iPSCs), such as human iPSCs. In some embodiments, the cells can be hematopoietic stem cells (HSCs), such as human HSCs. In some embodiments, the cells can be embryonic stem cells (ES cells), such as mouse ES cells or rat ES cells. In some embodiments, the cells can be one-cell stage embryos, such as mouse one-cell stage embryos or rat one-cell stage embryos.

In some cases, the cells comprising the targeted genetic modification made by the methods disclosed herein can be used to make a genetically modified organism comprising the targeted genetic modification. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214(2-3):91-109, each of which is herein incorporated by reference in its entirety for all purposes.

For example, the method of producing a non-human animal comprising the targeted genetic modification at the target genomic locus can comprise: (1) modifying the genome of a pluripotent cell to comprise the targeted genetic modification at the target genomic locus; (2) identifying or selecting the genetically modified pluripotent cell comprising the targeted genetic modification at the target genomic locus; (3) introducing the genetically modified pluripotent cell into a non-human animal host embryo; and (4) gestating the host embryo in a surrogate mother. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the targeted genetic modification at the target genomic locus.

An example of a suitable pluripotent cell is an embryonic stem (ES) cell (e.g., a mouse ES cell or a rat ES cell). The modified pluripotent cell can be generated, for example, using the methods disclosed herein. The donor cell can be introduced into a host embryo at any stage, such as the blastocyst stage or the pre-morula stage (i.e., the 4 cell stage or the 8 cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the targeted genetic modification at the target genomic locus; (2) selecting the genetically modified embryo; and (3) gestating the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise targeted genetic modification at the target genomic locus. It is recognized that depending on the method used to generate the F0 animal, the number of cells within the F0 animal that have targeted genetic modification at the target genomic locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for targeted genetic modification at the target genomic locus or can be homozygous for targeted genetic modification at the target genomic locus.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

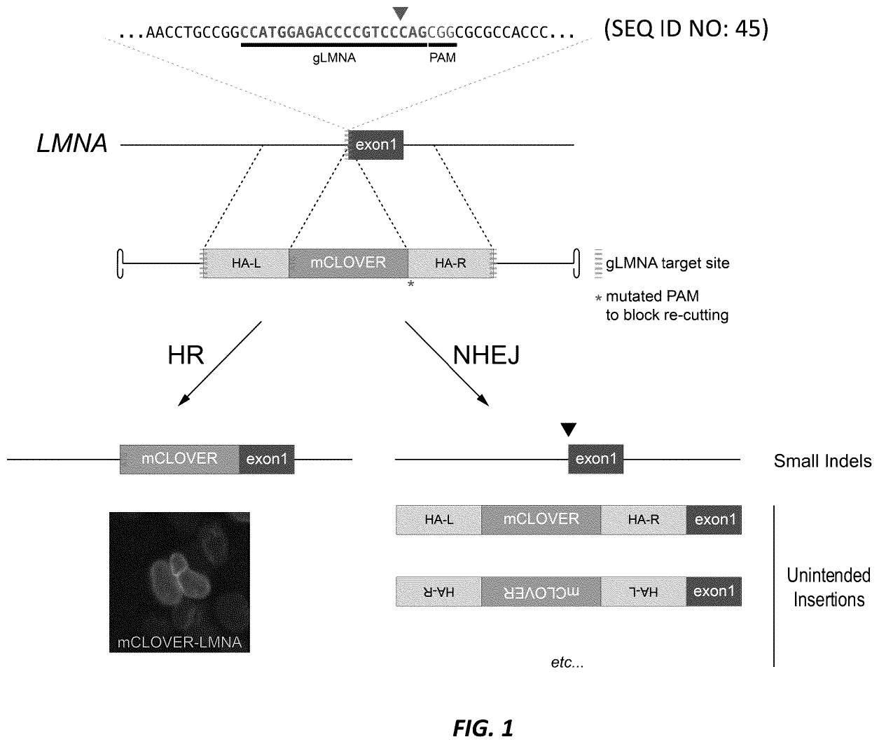

TABLE 2
Description of Sequences.

| SEQ ID NO | Type | Description |
|---|---|---|
| 1 | Protein | Cas9 protein |
| 2 | DNA | Cas9 protein CDS |
| 3 | Protein | SV40 NLS 1 |
| 4 | Protein | SV40 NLS 2 |
| 5 | Protein | Nucleoplasmin NLS |
| 6 | RNA | crRNA 1 |
| 7 | RNA | crRNA 2 |
| 8 | RNA | tracrRNA 1 |
| 9 | RNA | tracrRNA 2 |
| 10 | RNA | tracrRNA 3 |
| 11 | RNA | Guide RNA scaffold 1 |
| 12 | RNA | Guide RNA scaffold 2 |
| 13 | RNA | Guide RNA scaffold 3 |
| 14 | RNA | Guide RNA scaffold 4 |
| 15 | RNA | Guide RNA scaffold 5 |
| 16 | RNA | Guide RNA scaffold 6 |
| 17 | RNA | Guide RNA scaffold 7 |
| 18 | RNA | Guide RNA scaffold 8 |
| 19 | RNA | MS2-binding loop |
| 20 | RNA | MS2-binding aptamer |
| 21 | RNA | Guide RNA scaffold with MS2-binding loops 1 |
| 22 | RNA | Guide RNA scaffold with MS2-binding loops 2 |
| 23 | RNA | Guide RNA scaffold with MS2-binding loops 3 |
| 24 | RNA | Guide RNA with MS2-binding loops 1 |
| 25 | RNA | Guide RNA with MS2-binding loops 2 |
| 26 | RNA | Guide RNA with MS2-binding loops 3 |
| 27 | RNA | LMNA guide RNA |
| 28 | Protein | Linker 1 |
| 29 | Protein | Linker 2 |
| 30 | Protein | MS2-CtIP fusion protein |
| 31 | DNA | MS2-CtIP fusion protein CDS |
| 32 | Protein | MS2 coat protein |
| 33 | DNA | MS2 coat protein CDS |
| 34 | Protein | Human CtIP (AAC14371.1) |
| 35 | DNA | cDNA encoding human CtIP (U72066.1) |
| 36 | DNA | Human CtIP CDS (U72066.1) |
| 37 | Protein | Human CtIP (Q99708-1, NP_002885.1) |
| 38 | DNA | cDNA encoding human CtIP (NM_002894.3) |
| 39 | DNA | Human CtIP CDS (CCDS11875.1) |
| 40 | Protein | i53 protein |
| 41 | DNA | i53 protein CDS |
| 42 | Protein | i53-FLAG protein |
| 43 | DNA | i53-FLAG protein CDS |
| 44 | DNA | mCLOVER donor |
| 45 | DNA | Sequence from FIG. 1 |

### EXAMPLES

### Example 1. Combinatorial Expression of DNA Repair Regulators Enhances CRISPR-Mediated Homologous Recombination

To stimulate homology-directed repair (HDR) without inhibiting the endogenous functions of other DSB repair machineries or cell cycle regulation, we generated a HDR booster cocktail containing mRNAs encoding the CtBP-interacting protein (CtIP) and the inhibitor of TP53-binding protein 1 (53BP1), namely i53. The first is involved in initiating DNA resection at the DSB site, the limiting step of HDR, whereas the latter inhibits 53BP1 recruitment to and function at damaged chromatin, in turn creating a favorable environment for HDR. We designed a dual delivery system able to deliver the combination of the two proteins together efficiently and transiently together with Cas9 mRNA, sgRNA, and donor template. In one version, the mRNAs are all packaged in the lipid nanoparticles (LNPs), while the donor template and sgRNA are delivered by an adeno-associated virus (AAV) vector. As shown below, our data indicate that the combination of two enhancers (MS2-CtIP and i53) offers significant improvement of CRISPR-mediated HDR by ˜20-fold in human cells, with fast, transient, and integration-free expression of these proteins, thus allowing a better and more efficient way to perform precise genome editing.

High-Throughput Microscopy-Based Assessment of CRISPR-Mediated HDR Efficiency Via Precise Knock-In of mCLOVER at the LMNA Locus.

To measure CRISPR-induced HDR efficiency, we used a microscopy-based assay to quantify the percentage of cells that have undergone precise gene integration. CRISPR reagents were designed to integrate the mCLOVER coding sequence into the 5′-end of the LMNA gene, which encodes the lamin A/C proteins. See FIG. 1. We targeted this gene because lamin A/C are distinctly localized in the nuclear envelope, allowing identification of cells that had undergone HDR (i.e., the in-frame expression of mCLOVER-lamin fusion). We designed a donor template (SEQ ID NO: 44) containing the mCLOVER coding sequence flanked by homology arms of about 600 bp corresponding to the regions flanking the LMNA start codon. To prevent re-cutting by Cas9 after integration, the repair template contained a silent mutation at the gRNA PAM sequence. The repair template was flanked by the gRNA target site, allowing the template to be linearized upon cleavage by Cas9 in the cell.

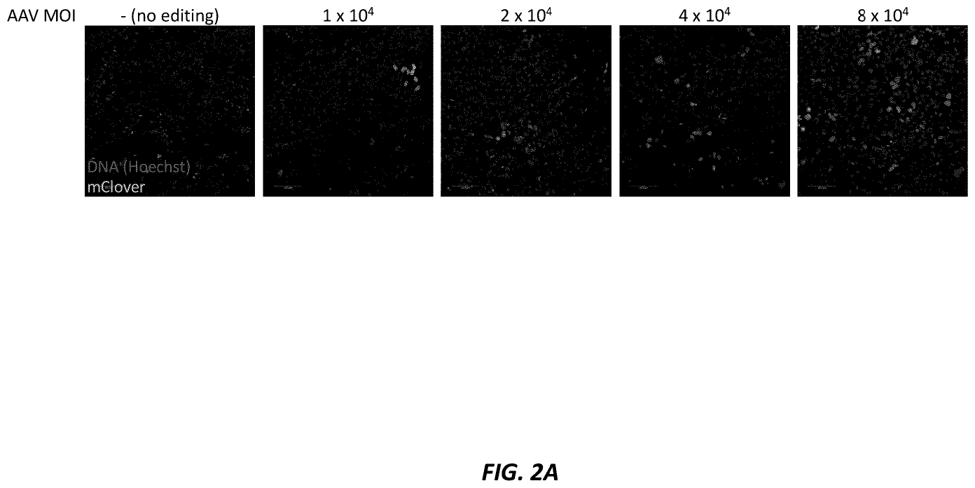

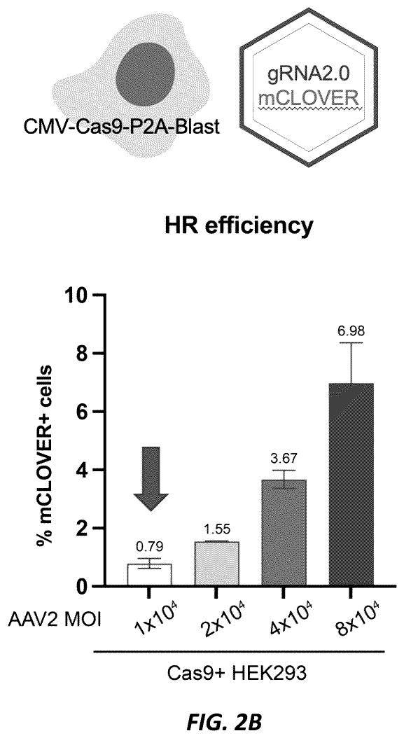

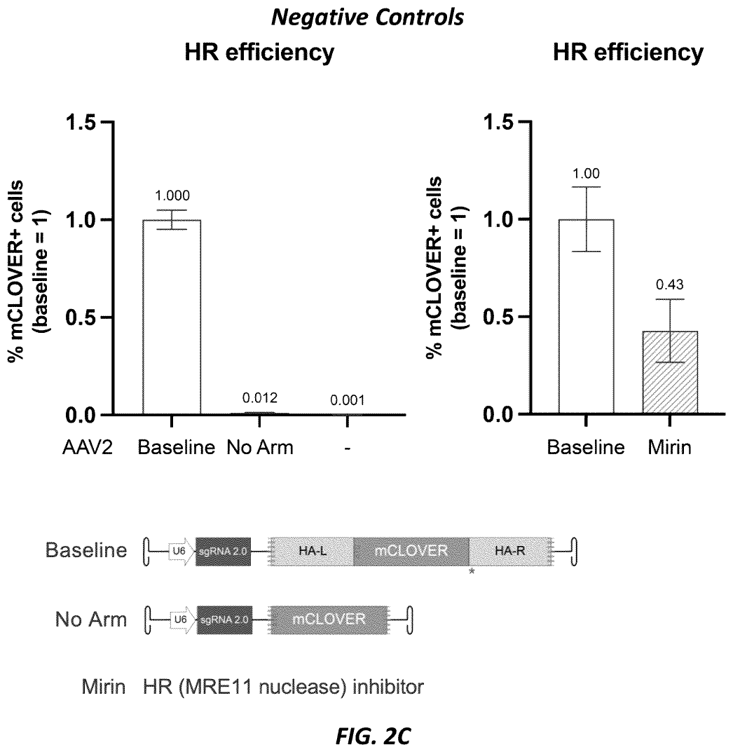

To enable LMNA-HDR, we transduced Cas9-expressing HEK293 cells with AAV2 packaged with vector containing the donor template as well as a U6-sgRNA2.0 expression cassette (AAV-sgRNA2.0-mClover). The Cas9 protein and DNA sequences are set forth in SEQ ID NOS: 1 and 2, respectively, and the LMNA sgRNA sequence is set forth in SEQ ID NO: 27. To optimize AAV delivery and to establish baseline HDR efficiency in these cells, we performed the transduction at different MOIs and imaged the cells by confocal microscopy 96h later. We observed increasing HDR events upon increasing sgRNA and donor template availability, validating the sensitivity of our assay. See FIGS. 2A-2B. AAV transduction into Cas9-expressing HEK293 cells (Cas9-HEK) at increasing MOIs led to a corresponding increase in the percentage of mClover-LMNA positive cells (FIG. 2B). The expression of mClover-LMNA was reduced when cells were co-treated with mirin, a well-documented MRE11, a homologous recombination protein, inhibitor, or when a donor template without HAs was used (FIG. 2C). These data suggest that the presence of mClover-LMNA fusion proteins indeed represents successful HDR.

### Screen of Homologous Recombination Proteins to Improve Efficiency of Cas9-Induced HDR.

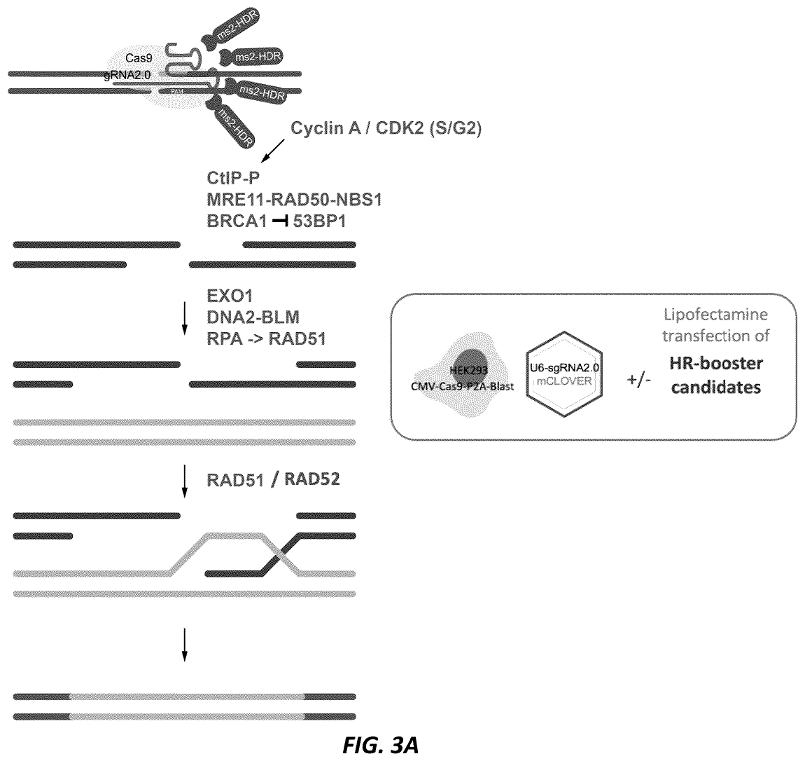

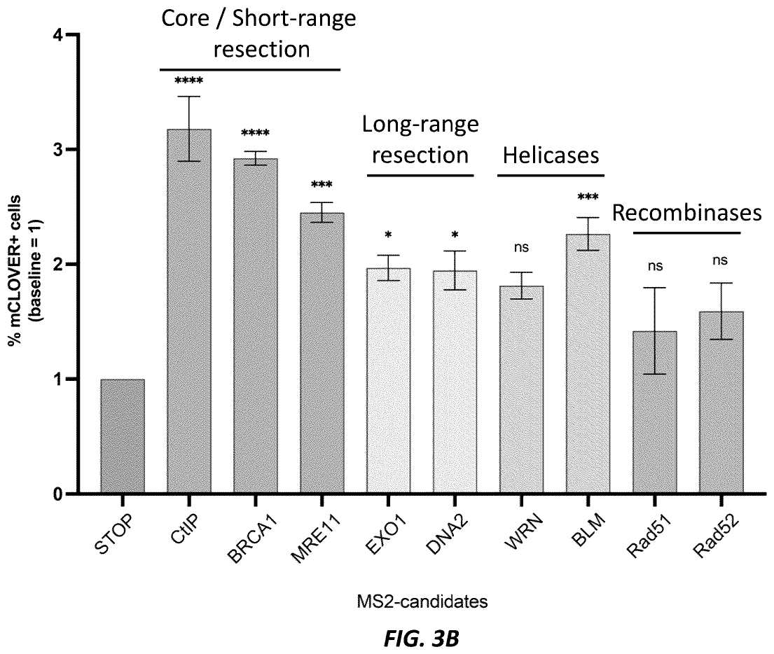

To selectively favor HDR repair at CRISPR-mediated DSBs, we aimed to identify and recruit potential HDR-boosting factors to Cas9 cleavage sites using the MS2-tagging approach. In this approach, the scaffold sequence of an sgRNA was modified to include MS2 phage aptamers (sgRNA2.0), and the HDR-boosting proteins were fused to the MS2 coat protein (MS2) for interaction with sgRNA2.0 (FIG. 3A). We hypothesized that the accumulation of these factors specifically at CRISPR/Cas9-targeted loci would increase the frequency of precision editing without altering the global DSB repair landscape. In this study, all sgRNAs used contained the MS2 scaffold unless specified otherwise.

We selected nine homologous recombination proteins and evaluated their effects on HDR frequency upon expression and recruitment to the Cas9-targeted LMNA locus. To do so, we generated individual plasmids expressing these proteins fused with MS2 at the 5′ ends. We then delivered the plasmids and AAV_{sgLMNA+mClover} simultaneously to Cas9-HEK cells and determined their respective HDR efficiency 96 hours later. While most candidates led to an increase in HDR, MS2-CtIP expression resulted in the most significant HDR improvement when compared to basal HDR activities induced by AAV_{sgLMNA+mClover} and an MS2-only plasmid (FIG. 3B).

### LNP Delivery of Individual End Resection Enhancers Improve Efficiency of Cas9-Induced HDR.

The CtIP protein plays a crucial role in DNA repair, particularly in DNA end resection, which is the rate-limiting step in homologous recombination. Despite its convenience and simplicity, plasmid delivery is limited to in vitro applications due to potential risks for immune response and non-specific DNA recombination with the genome. In addition, while CtIP is generally considered a tumor suppressor, studies have indicated its potential oncogenic role in facilitating tumorigenesis. In particular, CtIP overexpression was found in gastric cancer; its amplification was also documented in several other cancers. See, e.g., Mozaffari et al. (2021) Semin. Cell. Dev. Biol. 113:47-56, herein incorporated by reference in its entirety for all purposes. Therefore, we sought to explore therapeutically relevant approaches for delivering exogenous MS2-CtIP to enhance precision editing while minimizing its unwanted prolonged activities. Lipid nanoparticles (LNPs) have emerged as an effective, clinically viable delivery method for CRISPR editing systems (e.g., for delivering Cas9 mRNA and gRNA) due to their ability to effectively encapsulate and deliver these components into cells. This approach enables rapid and transient expression of Cas9 in cells, allowing for efficient editing while mitigating concerns associated with long-term, off-target nuclease exposure. Based on this, we proposed that the delivery of MS2-CtIP mRNA through LNP could result in efficient HDR-boosting activity, without any lasting negative effects.

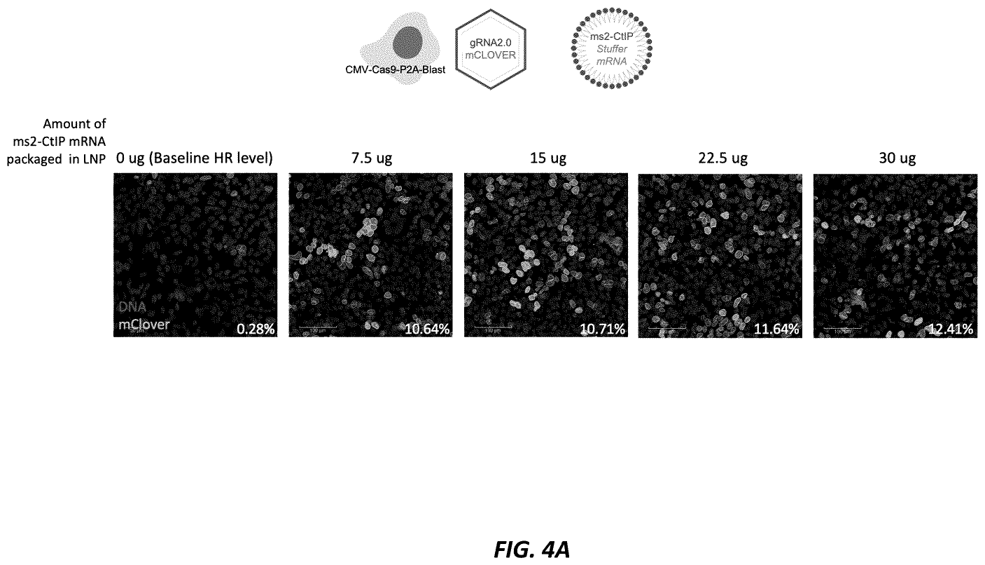

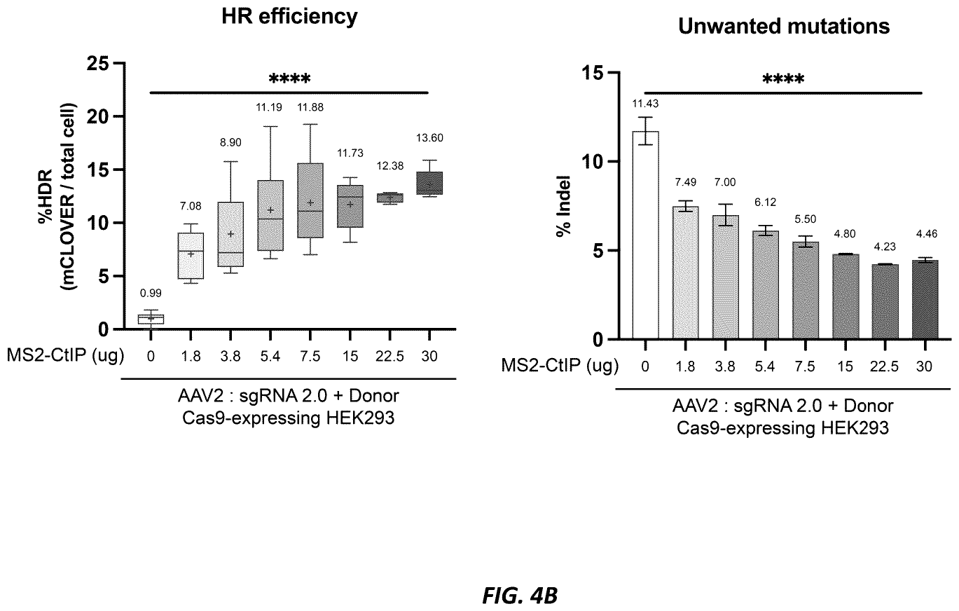

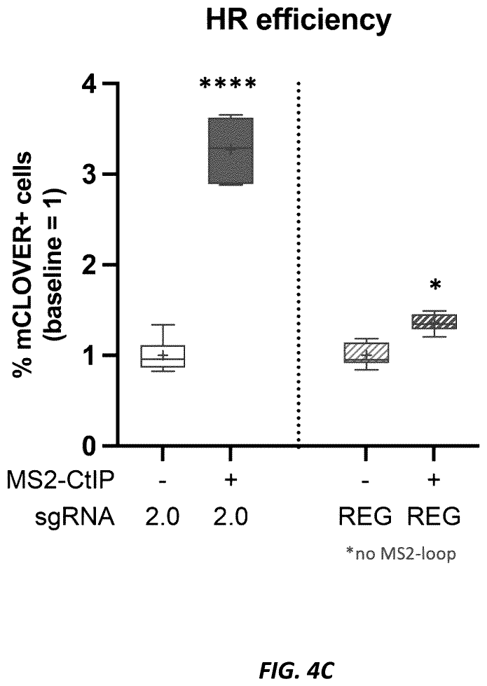

To test whether CtIP localization can boost HDR, we tested whether LNP-mediated delivery of MS2-CtIP mRNA (LNP-MS2-CtIP) (amino acid and nucleotide sequences set forth in SEQ ID NOS: 30 and 31, respectively) can influence HDR efficiency in Cas9-HEK293. We simultaneously delivered LNP-MS2-CtIP and AAV-sgRNA2.0-mClover to Cas9-HEK293 cells (FIG. 4A). To maximize the resolution of the effect of LNP-MS2-CtIP, we transduced the cells with AAV at the lowest MOI we have tested (1×10⁴, resulting in 0.28% of HDR in one representative replicate), and in parallel treated cells with LNP packaged with various amounts of mRNA. Our results indicated that the expression of MS2-CtIP leads to up to a ˜13-fold increase of HDR at the LMNA locus (FIG. 4B). Co-delivery of MS2-CtIP with AAV containing the donor template and regular gRNA without the aptamer (AAV-sgRNA-mClover) did not lead to any changes in HDR efficiency (FIG. 4C), suggesting that the localization of CLIP to the DSBs is required to commit the cell to repair the break via HDR. Next, we analyzed the indel rate at the LMNA locus of the same samples using NGS amplicon sequencing to quantify the frequency of insertional or deletional mutations (indels) surrounding the Cas9 cleavage site and found that MS2-CtIP treatment reduced the efficiency of indel formation, suggesting that end resection induced by MS2-CtIP shifts the DSB repair balance from NHEJ to HDR (FIG. 4B). Our result indicates that the enhanced availability of CtIP at CRISPR sites can significantly increase the efficiency of precise gene knock-in. See FIGS. 4A-4C. Collectively, our data suggested that localizing the CtIP protein to the Cas9 cleavage site can effectively shift the repair pathway choice from NHEJ to HDR, and that LNP transfection is a suitable delivery method for transient MS2-CtIP expression.

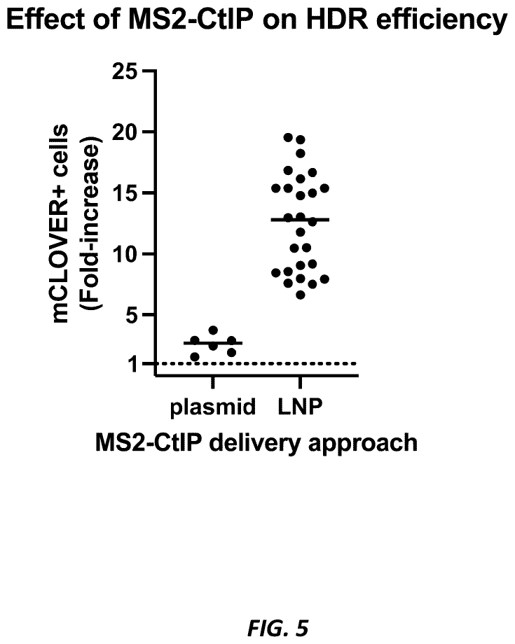

We next compared LNP delivery of exogenous MS2-CtIP mRNA to plasmid delivery of MST-CHIP DNA and found that LNP delivery of mRNA worked better than plasmid expression. The transient nature of the expression of MS2-CtIP through LNP-mediated delivery raised concerns about its potential to diminish the efficacy in enhancing HDR, compared to if MS2-CtIP were stably expressed such as from a plasmid. Contrary to our expectations, when we compared the effect of MS2-CLIP on HDR efficiency delivered via LNP versus plasmid expression, we observed a significantly stronger effect with LNP delivery. Specifically, there was a 12.5-fold increase in HDR efficiency with LNP, compared to a 2.5-fold increase with plasmid expression, relative to the baseline HDR efficiency when no MS2-CtIP was delivered (FIG. 5).

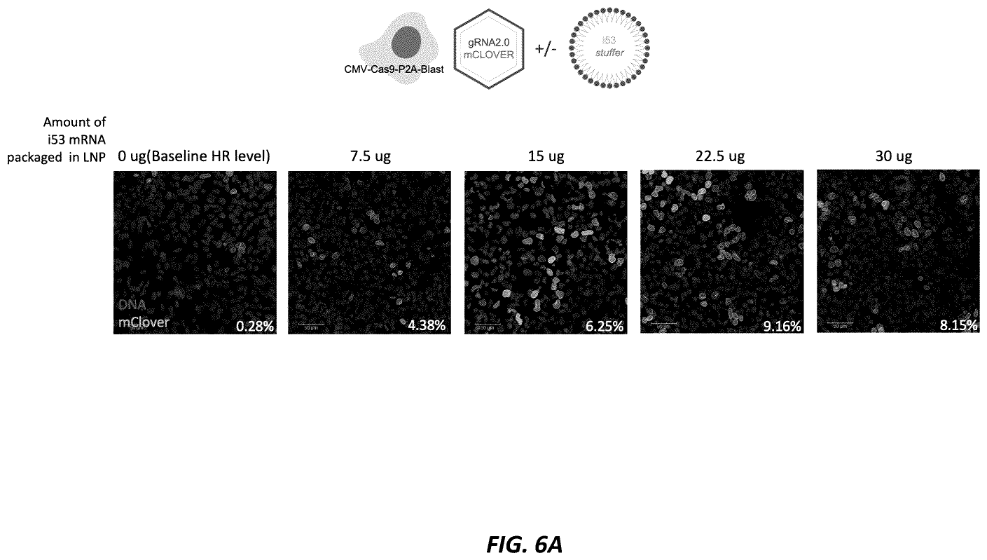

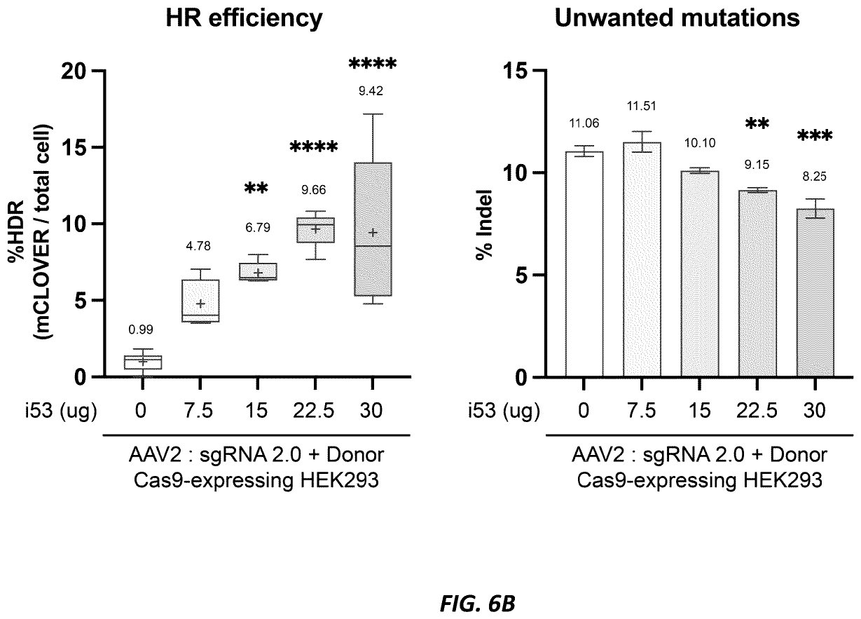

Given that the LNP-mediated enhancement of end resection can promote CRISPR-HDR, we next assessed whether the expression of an end protection inhibitor, namely the 53BP1-inhibiting-peptide i53, can also promote HDR upon CRISPR editing. 53BP1 plays a key role in DSB repair pathway choice by preventing the early step of DNA resection, in part by physically blocking resection nuclease activities and by mediating CtIP dephosphorylation. We packaged various amount of i53 mRNA (amino acid and nucleotide sequences set forth in SEQ ID NOS: 42 and 43, respectively) in LNP and delivered them to the Cas9-HEK293 cells treated with AAV (FIG. 6A). Increasing i53 expression results in a gradual and significant increase in HDR (FIG. 6B). Correspondingly, we observed a reduction in indel rate at the LMNA locus, indicating the LNP-mediated expression of i53 can promote HDR upon CRISPR editing (FIG. 6B). Our data indicate that 53BP1 inhibition, which indirectly allows for BRCA1 function and DNA end resection, is a feasible approach to boost CRISPR-mediated precising gene knock-in. See FIGS. 6A-6B.

### Combinatorial Expression of i53 and MS2-CtiP Further Enhances HDR Rates

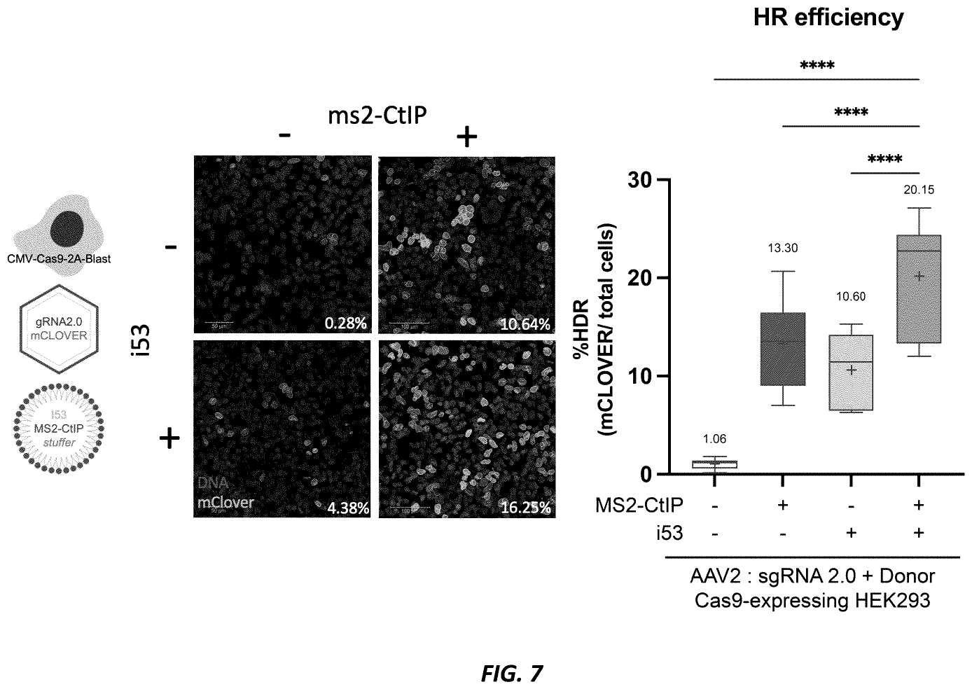

We reasoned that while recombinant CtIP overexpression and localization to CRISPR site can directly enhance DNA end resection for HDR activation, the role of 53BP1 inhibition is to indirectly provide an HDR-permissive environment for CtIP and other HDR proteins to function, without significantly downregulating NHEJ. Therefore, we tested whether co-delivering MS2-CtIP and i53 mRNA via LNP could induce a combinatorial HDR enhancement effect in CRISPR-treated cells. We treated Cas9-expressing HEK293 cells with AAV-sgRNA2.0-mClover and LNP encapsulating both MS2-CtIP and i53. Our data suggested that delivering 7.5 μg of MS2-CtIP and 15 μg of i53 via LNP in CRISPR-treated cells can enhance HDR efficiency ˜20-fold in HEK cells. See FIG. 7. Combinatorial enhancement of HDR efficiency was also observed in Cas9-expressing Huh7 cells, with a ˜5-fold enhancement of HDR efficiency over baseline (data not shown).

It is known that during S/G2 phase of cell cycle (when HDR is permissive), CtIP gets phosphorylated by cell cycle regulator CDKs, which in turn inhibits 53BP1 function and initiates HDR. See, e.g., Daley et al. (2014) Mol. Cell. Biol. 34(8):1380-1388 (“In S/G₂, CtIP is phosphorylated by CDK, inducing the formation of a complex with BRCA1 and MRN. This complex displaces 53BP1 and initiates resection.”), herein incorporated by reference in its entirety for all purposes. Given this well-established mechanism, it was surprising to find that in our system, the addition of i53 alongside exogenous MS2-CtIP overexpression/localization could further increase HDR efficiency. It has been previously reported that silencing 53BP1 or exhausting its capacity to bind damaged chromatin changes limited DSB resection to hyper-resection and results in a switch from error-free gene conversion by RAD51 to mutagenic single-strand annealing by RAD52. See, e.g., Ochs et al. (2016) Nat. Struct. Mol. Biol. 23(8):714-721, herein incorporated by reference in its entirety for all purposes. Based on this, one would speculate that our booster, which employs two strategies prone to resection, might result in the mutagenic editing outcomes indicated by the study (hyper-resection). However, contrary to this expectation, our booster successfully promoted error-free HDR.

Our strategy, to our knowledge, results in the highest HDR enhancement rate compared to other published strategies. Our strategy offers potential benefits in various research applications such as disease model generation in cells and in vivo, as well as in therapeutic settings in which the efficiency of precise gene knock-in may be significantly enhanced. Our strategy also offers benefits over approaches in which all components are fused to a single Cas9 protein. For example, our system facilitates additional recruitment of CtIP molecules, which has been described to work as multimeric complex, whereas a fusion protein would allow only one CtIP molecule as only one Cas9 molecule can bind to the target site. Similarly, i53 will likely have a more robust effect when delivered independently than as a fusion with limited stoichiometry.

### Chemical Inhibition of NHEJ

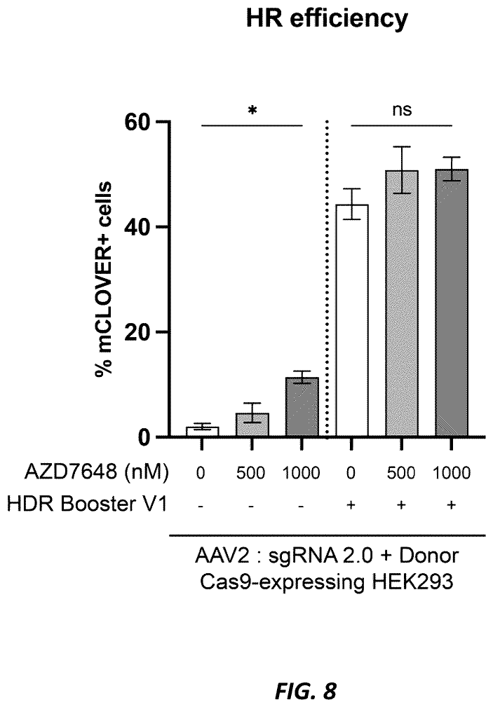

Chemical inhibition of NHEJ is often employed as a strategy to enhance HDR in cells. In this context, we compared our HDR booster with AZD7648, one of the most potent NHEJ (DNA-PKcs) inhibiting small molecules. Our reporter indicated that the HDR booster significantly outperformed AZD7648 in enhancing HDR (FIG. 8). Notably, the combination of the HDR booster and AZD7648 did not result in further HDR enhancement (FIG. 8). We reasoned that our HDR booster has maximized CRISPR-mediated HDR at the LMNA locus, given the amount of sgRNA/donor template provided and the capacity of the endogenous HDR machinery.

HDR Booster Achieves Precise Gene Integration with Reducing HDR Template

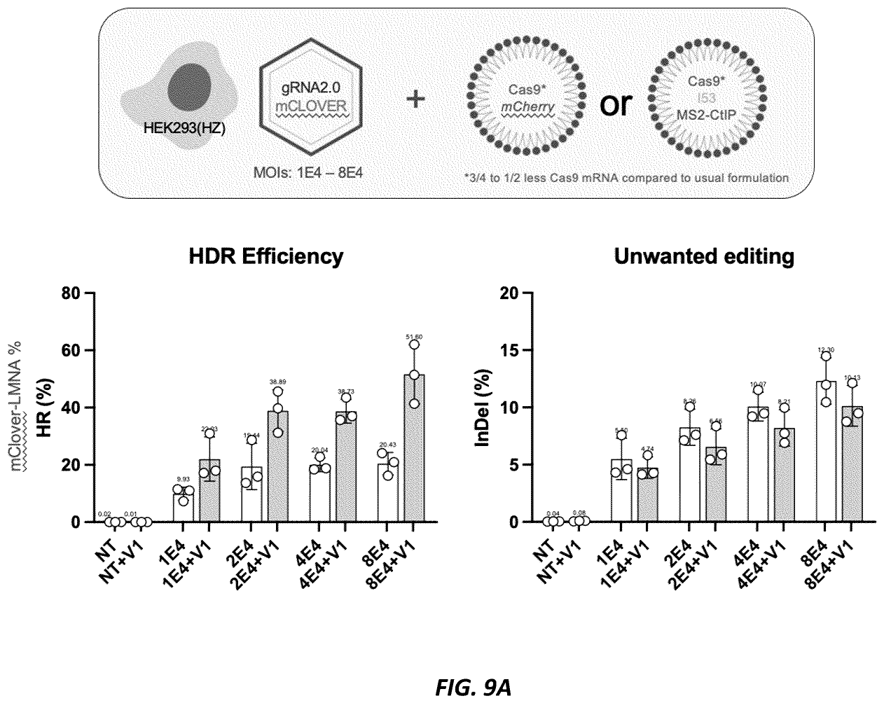

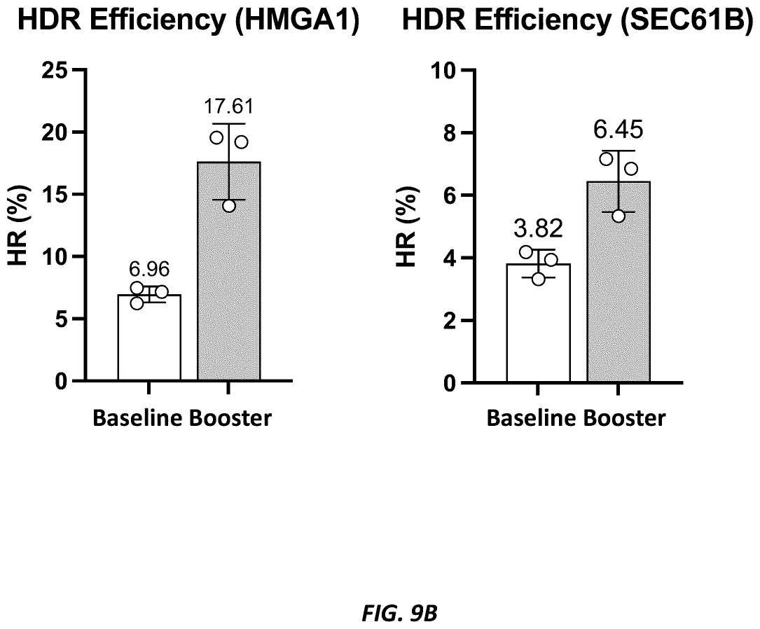

To mimic therapeutic gene editing, we next investigated the effect of HDR booster in precise gene editing when Cas9 is also transiently expressed by LNP, rather than already being stably expressed in a cell line. Concurrently, we tested whether HDR booster allows for a reduction in the amount of AAV_{sgLMNA+mClover} without affecting gene targeting efficiency. To this end, we prepared two LNPs: LNP_{booster} which encapsulates Cas9, MS2-CtIP, and i53 mRNAs, and LNP_{baseline} which encapsulates Cas9 and mCherry mRNAs. We then transfected the LNPs into HEK293 cells that were transduced with different MOIs of AAV_{sgLMNA+mClover}. As expected, lowering AAV MOIs resulted in a decrease in gene targeting efficiency. Significantly, regardless of the AAV MOI, we observed a consistent increase in HDR efficiency in cells treated with LNP_{booster} compared to those treated with LNP_{baseline} (FIG. 9A). This was accompanied by a corresponding decrease in indel formation (FIG. 9A).

We repeated the experiment targeting the C-terminus of two other genes, HMGA1 and SEC61B, and reached a similar conclusion (FIG. 9B). Together, our results suggest that HDR booster can be used in a clinically relevant setting in which Cas9 is co-expressed transiently with the booster. Importantly, this codelivery allows for high precision gene integration efficiency while reducing the amounts of CRISPR reagents (AAV) needed, thus reducing the opportunities of off-target CRISPR activities and transgene integration.

### HDR Booster Promotes Precise Gene Integration Using Different Delivery Approaches

The above data highlights the capacity of the HDR booster to enhance precise editing using the LNP/AAV co-delivery approach. While AAV remains the preferred choice for donor template carriers in in vivo preclinical and clinical settings, non-viral templates such as linear dsDNA and ssDNA are emerging as promising alternatives. This is particularly evident in the gene editing of clinically relevant primary human hematopoietic cells, as well as in CRIPSR-mediated disease modeling using iPSCs, mESCs, and animal embryos.

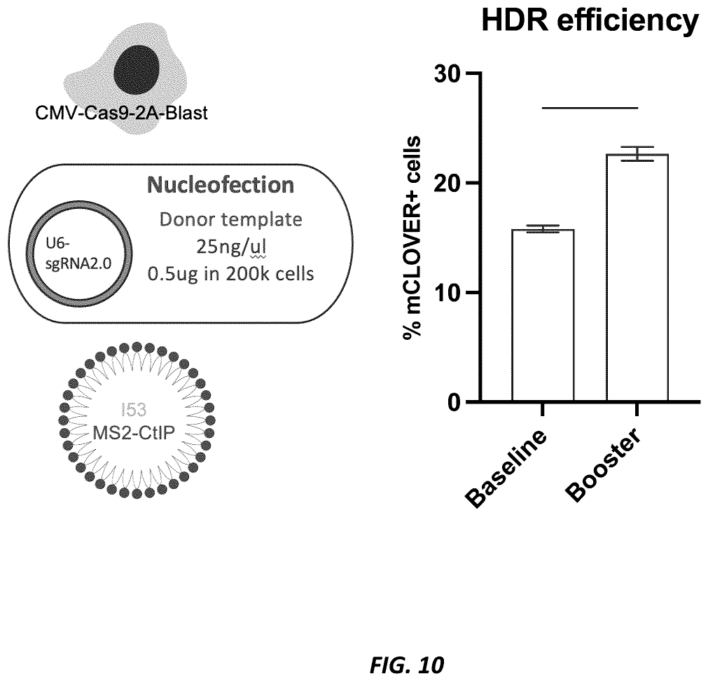

We therefore sought to investigate whether the HDR booster can enhance HDR in the context of non-viral donor delivery. To achieve this, we obtained linear closed-ended dsDNA containing mClover coding sequence flanked by LMNA homology arm sequences as described above (dsDNA_{mclover-LMNA}) and delivered it into Cas9-HEK cells along with plasmid encoding sgLMNA2.0 via electroporation. Electroporated cells were then cultured in media with or without HDR booster encapsulated LNP for 96 hours. Our data showed that the inclusion of HDR booster significantly increase the percentage of mClover-LMNA cells (FIG. 10).

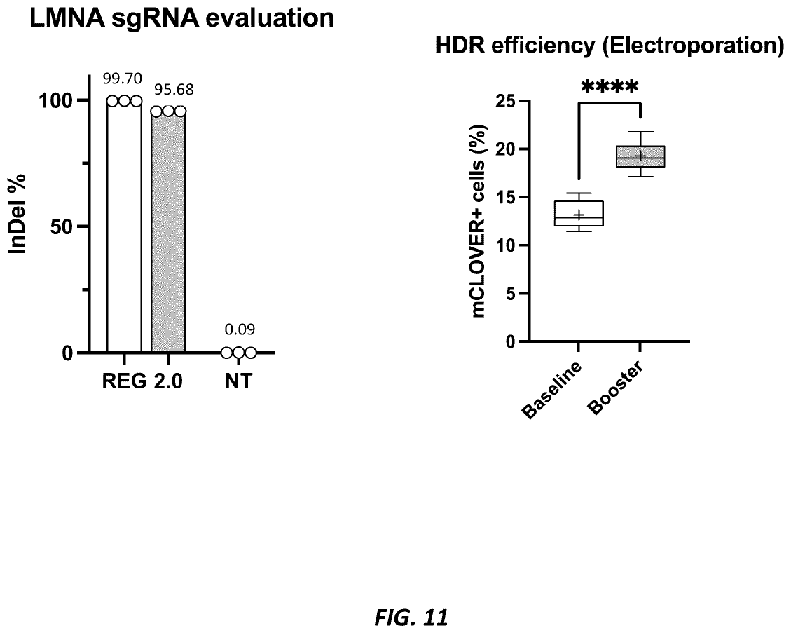

We also tested whether the HDR booster mRNA could be co-delivered with rest of the CRISPR reagents via electroporation. To this end, we obtained synthetic sgLMNA2.0 and validated that their editing efficiency was as good as synthetic sgLMNA-REG. We then electroporated sgLMNA2.0, dsDNA_{mclover-LMNA}, and mRNAs of HDR booster into HEK-Cas9, and observed a significant increase in HDR efficiency (FIG. 11). Together, our findings demonstrates that the HDR booster can be effectively paired with different delivery approaches, underlining its extensive versatility.

1. A method for making a targeted genetic modification by homology-directed repair at a target genomic locus in a cell, comprising administering to the cell:
(a) a Clustered Regularly Interspaced Short Palindromic Repeats (CRISPR) associated (Cas) protein or a nucleic acid encoding the Cas protein;
(b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus;
(c) a fusion protein or a nucleic acid encoding the fusion protein, wherein the fusion protein comprises a CtBP-interacting protein (CtIP) fused to the adaptor protein;
(d) an inhibitor of 53BP1 (153) protein or a nucleic acid encoding the i53 protein; and
(e) an exogenous donor nucleic acid comprising a 5′ homology arm that hybridizes to a 5′ target sequence at the target genomic locus and a 3′ homology arm that hybridizes to a 3′ target sequence at the target genomic locus, optionally wherein the 5′ homology arm and the 3′ homology arm flank an insert nucleic acid,
wherein the Cas protein and the guide RNA form a complex, the Cas protein cleaves the guide RNA target sequence to create a double-strand break, and the exogenous donor nucleic acid recombines with the target genomic locus via homology-directed repair to create the targeted genetic modification.

2. The method of claim 1, wherein the Cas protein is administered to the cell in the form of a protein, optionally wherein the Cas protein is in a lipid nanoparticle.

3. The method of claim 1, wherein the nucleic acid encoding the Cas protein is administered to the cell, wherein the nucleic acid encoding the Cas protein comprises an RNA encoding the Cas protein, optionally wherein the RNA encoding the Cas protein is in a lipid nanoparticle.

4. The method of claim 1, wherein the nucleic acid encoding the Cas protein is administered to the cell, wherein the nucleic acid encoding the Cas protein comprises a DNA encoding the Cas protein, optionally wherein the DNA encoding the Cas protein is in a viral vector, optionally wherein the viral vector is a recombinant adeno-associated virus (AAV) vector.

5. The method of claim 1, wherein the Cas protein is a Cas9 protein.

6. The method of claim 5, wherein the Cas9 protein is a Streptococcus pyogenes Cas9 protein, a Campylobacter jejuni Cas9 protein, or a Staphylococcus aureus Cas9 protein, optionally wherein the Cas9 protein is the Streptococcus pyogenes Cas9 protein.

7. The method of claim 1, wherein the guide RNA is administered in the form of RNA, optionally wherein the guide RNA is in a lipid nanoparticle.

8. The method of claim 1, wherein the one or more DNAs encoding the guide RNA are administered to the cell, optionally wherein the one or more DNAs encoding the guide RNA are in a viral vector, optionally wherein the viral vector is a recombinant AAV vector.

9. The method of claim 1, wherein the guide RNA comprises two adaptor-binding elements to which the adaptor protein can specifically bind.

10. The method of claim 9, wherein a first adaptor-binding element is within a first loop of the guide RNA, and a second adaptor-binding element is within a second loop of the guide RNA.

11. The method of claim 10, wherein the guide RNA is a single guide RNA comprising a CRISPR RNA (crRNA) portion fused to a transactivating CRISPR RNA (tracrRNA) portion, and
wherein the first loop is the tetraloop corresponding to residues 13-16 of SEQ ID NO: 11, 13, 15, or 16, and the second loop is the stem loop 2 corresponding to residues 53-56 of SEQ ID NO: 11, 13, 15, or 16.

12. The method of claim 1, wherein the adaptor-binding element comprises the sequence set forth in SEQ ID NO: 19 or 20.

13. The method of claim 1, wherein the guide RNA comprises the sequence set forth in SEQ ID NO: 21, 22, 23, 24, 25, or 26.

14. The method of claim 1, wherein the fusion protein is administered to the cell in the form of a protein, optionally wherein the fusion protein is in a lipid nanoparticle.

15. The method of claim 1, wherein the nucleic acid encoding the fusion protein is administered to the cell, wherein the nucleic acid encoding the fusion protein comprises an RNA encoding the fusion protein, optionally wherein the RNA encoding the fusion protein is in a lipid nanoparticle.

16. The method of claim 1, wherein the nucleic acid encoding the fusion protein is administered to the cell, wherein the nucleic acid encoding the fusion protein comprises a DNA encoding the fusion protein, optionally wherein the DNA encoding the fusion protein is in a viral vector, optionally wherein the viral vector is a recombinant AAV vector.

17. The method of claim 1, wherein the adaptor protein comprises an MS2 coat protein or a functional fragment or variant thereof.

18. The method of claim 1, wherein the adaptor protein comprises a sequence at least 90%, 95%, 96%, 97%, 98%, 99%, or 100% identical to the sequence set forth in SEQ ID NO: 32.

19. The method of claim 1, wherein the adaptor protein is encoded by a sequence at least 90%, 95%, 96%, 97%, 98%, 99%, or 100% identical to the sequence set forth in SEQ ID NO: 33.

20. The method of claim 1, wherein the CtIP protein comprises a sequence at least 90%, 95%, 96%, 97%, 98%, 99%, or 100% identical to the sequence set forth in SEQ ID NO: 34.

21. The method of claim 1, wherein the CtIP protein is encoded by a sequence at least 90%, 95%, 96%, 97%, 98%, 99%, or 100% identical to the sequence set forth in SEQ ID NO: 36.

22. The method of claim 1, wherein the fusion protein comprises a sequence at least 90%, 95%, 96%, 97%, 98%, 99%, or 100% identical to the sequence set forth in SEQ ID NO: 30.

23. The method of claim 1, wherein the fusion protein is encoded by a sequence at least 90%, 95%, 96%, 97%, 98%, 99%, or 100% identical to the sequence set forth in SEQ ID NO: 31.

24. The method of claim 1, wherein the i53 protein is administered to the cell in the form of a protein, optionally wherein the i53 protein is in a lipid nanoparticle.

25. The method of claim 1, wherein the nucleic acid encoding the i53 protein is administered to the cell, wherein the nucleic acid encoding the i53 protein comprises an RNA messenger RNA encoding the i53 protein, optionally wherein the RNA encoding the i53 protein is in a lipid nanoparticle.

26. The method of claim 1, wherein the nucleic acid encoding the i53 protein is administered to the cell, wherein the nucleic acid encoding the i53 protein comprises a DNA encoding the i53 protein, optionally wherein the DNA encoding the i53 protein is in a viral vector, optionally wherein the viral vector is a recombinant AAV vector.

27. The method of claim 1, wherein the i53 protein comprises a sequence at least 90%, 95%, 96%, 97%, 98%, 99%, or 100% identical to the sequence set forth in SEQ ID NO: 40 or 42.

28. The method of claim 1, wherein the i53 protein is encoded by a sequence at least 90%, 95%, 96%, 97%, 98%, 99%, or 100% identical to the sequence set forth in SEQ ID NO: 41 or 43.

29. The method of claim 1, wherein the exogenous donor nucleic acid comprises the insert nucleic acid.

30. The method of claim 1, wherein the exogenous donor nucleic acid is in a viral vector.

31. The method of claim 30, wherein the viral vector is a recombinant AAV vector.

32. The method of claim 1, wherein the exogenous donor nucleic acid is a large targeting vector (LTVEC), wherein:
(a) the LTVEC is at least 10 kb;
(b) the sum total of the 5′ and 3′ homology arms of the LTVEC is at least 10 kb;
(c) the LTVEC is from about 50 kb to about 300 kb; or
(d) the sum total of the 5′ and 3′ homology arms of the LTVEC is from about 10 kb to about 200 kb.

33. The method of claim 1, wherein the nucleic acid encoding the fusion protein is administered to the cell, wherein the nucleic acid encoding the fusion protein comprises an RNA encoding the fusion protein,
wherein the nucleic acid encoding the i53 protein is administered to the cell, wherein the nucleic acid encoding the i53 protein comprises an RNA encoding the i53 protein, and
wherein the RNA encoding the fusion protein and the RNA encoding the i53 protein are in a lipid nanoparticle.

34. The method of claim 1, wherein the guide RNA comprises two adaptor-binding elements to which the adaptor protein can specifically bind, wherein a first adaptor-binding element is within a first loop of the guide RNA, and a second adaptor-binding element is within a second loop of the guide RNA,
wherein the adaptor protein comprises an MS2 coat protein or a functional fragment or variant thereof,
wherein the nucleic acid encoding the fusion protein is administered to the cell, wherein the nucleic acid encoding the fusion protein comprises an RNA encoding the fusion protein,
wherein the nucleic acid encoding the i53 protein is administered to the cell, wherein the nucleic acid encoding the i53 protein comprises an RNA encoding the i53 protein, and
wherein the RNA encoding the fusion protein and the RNA encoding the i53 protein are in a lipid nanoparticle.

35. The method of claim 1, wherein the guide RNA comprises two adaptor-binding elements to which the adaptor protein can specifically bind, wherein a first adaptor-binding element is within a first loop of the guide RNA, and a second adaptor-binding element is within a second loop of the guide RNA,
wherein the adaptor protein comprises an MS2 coat protein or a functional fragment or variant thereof,
wherein the nucleic acid encoding the Cas protein is administered to the cell, wherein the nucleic acid encoding the Cas protein comprises an RNA encoding the Cas protein,
wherein the nucleic acid encoding the fusion protein is administered to the cell, wherein the nucleic acid encoding the fusion protein comprises an RNA encoding the fusion protein,
wherein the nucleic acid encoding the i53 protein is administered to the cell, wherein the nucleic acid encoding the i53 protein comprises an RNA encoding the i53 protein,
wherein the RNA encoding the Cas protein, the RNA encoding the fusion protein, and the RNA encoding the i53 protein are in a lipid nanoparticle,
wherein the one or more DNAs encoding the guide RNA are administered to the cell, and
wherein the one or more DNAs encoding the guide RNA and the exogenous donor nucleic acid are in a recombinant AAV vector.

36. The method of claim 1, wherein the guide RNA comprises two adaptor-binding elements to which the adaptor protein can specifically bind, wherein a first adaptor-binding element is within a first loop of the guide RNA, and a second adaptor-binding element is within a second loop of the guide RNA,
wherein the adaptor protein comprises an MS2 coat protein or a functional fragment or variant thereof,
wherein the nucleic acid encoding the Cas protein is administered to the cell, wherein the nucleic acid encoding the Cas protein comprises an RNA encoding the Cas protein,
wherein the nucleic acid encoding the fusion protein is administered to the cell, wherein the nucleic acid encoding the fusion protein comprises an RNA encoding the fusion protein,
wherein the nucleic acid encoding the i53 protein is administered to the cell, wherein the nucleic acid encoding the i53 protein comprises an RNA encoding the i53 protein,
wherein the guide RNA is administered to the cell in the form of RNA,
wherein the RNA encoding the Cas protein, the RNA encoding the fusion protein, the RNA encoding the i53 protein, and the guide RNA are in a lipid nanoparticle,
wherein the exogenous donor nucleic acid is in a recombinant AAV vector.

37. The method of claim 1, wherein the cell is a mammalian cell.

38. The method of claim 1, wherein the cell is a rodent cell.

39. The method of claim 1, wherein the cell is a mouse cell or a rat cell.

40. The method of claim 1, wherein the cell is a mouse cell.

41. The method of claim 1, wherein the cell is a human cell.

42. The method of claim 1, wherein the cell is in vitro.

43. The method of claim 1, wherein the cell is in vivo.

44. A composition or combination comprising:
(a) a Clustered Regularly Interspaced Short Palindromic Repeats (CRISPR) associated (Cas) protein or a nucleic acid encoding the Cas protein;
(b) a guide RNA or one or more DNAs encoding the guide RNA, wherein the guide RNA comprises one or more adaptor-binding elements to which an adaptor protein can specifically bind, and wherein the guide RNA is capable of forming a complex with the Cas protein and guiding it to a guide RNA target sequence at the target genomic locus;
(c) a fusion protein or a nucleic acid encoding the fusion protein, wherein the fusion protein comprises a CtBP-interacting protein (CtIP) fused to the adaptor protein;
(d) an inhibitor of 53BP1 (153) protein or a nucleic acid encoding the i53 protein; and
(e) an exogenous donor nucleic acid comprising a 5′ homology arm that hybridizes to a 5′ target sequence at the target genomic locus and a 3′ homology arm that hybridizes to a 3′ target sequence at the target genomic locus, optionally wherein the 5′ homology arm and the 3′ homology arm flank an insert nucleic acid.

45.-79. (canceled)


SEQUENCE LISTING (45 sequences)

SEQ ID NO: 1 - protein, 1392 aa, synthetic construct
  REGION 1369..1384: note = Nucleoplasmin Nuclear Localization Sequence
  REGION 1385..1392: note = FLAG tag
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKDYKDDDDK

SEQ ID NO: 2 - DNA, 4176 bp, synthetic construct
  source 1..4176: mol_type = other DNA
  misc_feature 4105..4152: note = Nucleoplasmin Nuclear Localization Sequence
  misc_feature 4153..4176: note = FLAG Tag
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataag

SEQ ID NO: 3 - protein, 7 aa, synthetic construct
PKKKRKV

SEQ ID NO: 4 - protein, 7 aa, synthetic construct
PKKKRRV

SEQ ID NO: 5 - protein, 16 aa, synthetic construct
KRPAATKKAGQAKKKK

SEQ ID NO: 6 - RNA, 16 nt, synthetic construct
  source 1..16: mol_type = other RNA
gttttagagctatgct

SEQ ID NO: 7 - RNA, 22 nt, synthetic construct
  source 1..22: mol_type = other RNA
gttttagagctatgctgttttg

SEQ ID NO: 8 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 9 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 10 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 11 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 12 - RNA, 82 nt, synthetic construct; identical to SEQ ID NO: 10
  source 1..82: mol_type = other RNA

SEQ ID NO: 13 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 14 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 15 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 16 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 17 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 18 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 19 - RNA, 34 nt, synthetic construct
  source 1..34: mol_type = other RNA
ggccaacatgaggatcacccatgtctgcagggcc

SEQ ID NO: 20 - RNA, 19 nt, synthetic construct
  source 1..19: mol_type = other RNA
acatgaggatcacccatgt

SEQ ID NO: 21 - RNA, 137 nt, synthetic construct
  source 1..137: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgct

SEQ ID NO: 22 - RNA, 139 nt, synthetic construct
  source 1..139: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 23 - RNA, 143 nt, synthetic construct
  source 1..143: mol_type = other RNA
gttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 24 - RNA, 157 nt, synthetic construct
  source 1..157: mol_type = other RNA
  misc_difference 1..20: note = n = a, u, c, or g
nnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgct

SEQ ID NO: 25 - RNA, 159 nt, synthetic construct
  source 1..159: mol_type = other RNA
  misc_difference 1..20: note = n = a, u, c, or g
nnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgcttt

SEQ ID NO: 26 - RNA, 163 nt, synthetic construct
  source 1..163: mol_type = other RNA
  misc_difference 1..20: note = n = a, u, c, or g
nnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 27 - RNA, 163 nt, synthetic construct
  source 1..163: mol_type = other RNA
ccatggagaccccgtcccaggttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 28 - protein, 4 aa, synthetic construct
GGGS

SEQ ID NO: 29 - protein, 5 aa, synthetic construct
GGGGS

SEQ ID NO: 30 - protein, 1055 aa, synthetic construct
  REGION 1..130: note = MS2 sequence
  REGION 131..148: note = poly-Glycine-Serine (G4S) linker - G4S linker
  REGION 149..155: note = SV40 NLS
  REGION 159..1055: note = hCtiP
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAMNILGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 31 - DNA, 3168 bp, synthetic construct
  source 1..3168: mol_type = other DNA
  misc_feature 1..390: note = MS2 sequence
  misc_feature 391..444: note = poly-Glycine-Serine (G4S) linker - G4S linker
  misc_feature 445..465: note = SV40 NLS
  misc_feature 475..3168: note = hCtiP
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctatgaacatcttgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 32 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 33 - DNA, 390 bp, synthetic construct
  source 1..390: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 34 - protein, 897 aa, Homo sapiens [sequence omitted]

SEQ ID NO: 35 - DNA, 3247 bp, Homo sapiens
  source 1..3247: mol_type = other DNA
cgggtccggccgctccgagcccggccgcagcccccggcttaaagcgcgggctgtccggagggtcggctttcccaccgaggatttggcactctggtgagggttttgggcgaaagagaaaagcgagcagccgtccttcacagcctcagaaagtgctcgcttcccttcggggctttcgcgaatcccgaggcaatctcggaggcggtatttgacctgtccaaagacgacttgatacctctataatgtaacagaaaaggtcagaaaatattaagcaagtagaagtgtggagcatattaagcaagatgaacatcttgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatagacgttgaaacagaaacagaaggatgaaggacagttttttccttcttagttatttatagttaaagttggtactaaacattgatttttttgatcttctgtaaatggatttataaatcagttttctattgaaaatgtttgtgatattttgcttttgcacctttaaaacaataaggcgctttcattttgcactctaacttaagagtttttactttatgtagtgatacctaatacaattttgaaaatacaaaaaaaaaa

SEQ ID NO: 36 - DNA, 2694 bp, Homo sapiens
  source 1..2694: mol_type = other DNA
atgaacatcttgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 37 - protein, 897 aa, Homo sapiens
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 38 - DNA, 3275 bp, Homo sapiens
  source 1..3275: mol_type = other DNA
gcgtgacgtcgcgcgggctcccgggcggggcgggtccggccgcctccgagcccggccggcagcccccggccttaaagcgcgggctgtccggaggggtcggctttcccaccgaggatttggcactctggtgagggaaaagggcgaaagagaaaagcgagcagccgtcctttcacagcctcagaaagtgctcgcttcccttcgggggctttcgcgaatcccgaggcaatctcggaggcggtatttgacctgtccaaagacgacttgatacctctataatgtaacagaaaaggtcagaaaatattaagcaagtagaagtgtggagcatattaagcaagatgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatagacgttgaaacagaaacagaaggatgaaggacagttttttccttcttagttatttatagttaaagttggtactaaacattgatttttttgatcttctgtaaatggatttataaatcagttttctattgaaaatgtttgtgatattttgcttttgcacctttaaaacaataaggcgctttcattttgcactctaacttaagagtttttactttatgtagtgatacctaatacaattttgaaaatacaa

SEQ ID NO: 39 - DNA, 2694 bp, Homo sapiens
  source 1..2694: mol_type = other DNA
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 40 - protein, 87 aa, synthetic construct
AASLNGAPLIKDPMLIFVKTLTGKTITLEVEPSDTIENVKAKIQDKEGIPPDQQRLAFAGKSLEDGRTLSDYNILKDSKLHPLLRLR

SEQ ID NO: 41 - DNA, 264 bp, synthetic construct
  source 1..264: mol_type = other DNA
gccgccagtttaaacggcgcgccattaattaaggatccaatgttgattttcgtgaaaacccttaccgggaaaaccatcaccctcgaggttgaaccctcggatacgatagaaaatgtaaaggccaagatccaggataaggaaggaattcctcctgatcagcagagactggcctttgctggcaaatcgctggaagatggacgtactttgtctgactacaatattctaaaggactctaaacttcatcctctgttgagacttcgttga

SEQ ID NO: 42 - protein, 96 aa, synthetic construct
MDYKDDDDKAASLNGAPLIKDPMLIFVKTLTGKTITLEVEPSDTIENVKAKIQDKEGIPPDQQRLAFAGKSLEDGRTLSDYNILKDSKLHPLLRLR

SEQ ID NO: 43 - DNA, 291 bp, synthetic construct
  source 1..291: mol_type = other DNA
atggactacaaagacgatgacgataaagccgccagtttaaacggcgcgccattaattaaggatccaatgttgattttcgtgaaaacccttaccgggaaaaccatcaccctcgaggttgaaccctcggatacgatagaaaatgtaaaggccaagatccaggataaggaaggaattcctcctgatcagcagagactggcctttgctggcaaatcgctggaagatggacgtactttgtctgactacaatattctaaaggactctaaacttcatcctctgttgagacttcgttga

SEQ ID NO: 44 - DNA, 1920 bp, synthetic construct
  source 1..1920: mol_type = other DNA
  misc_feature 1..23: note = Sequence recognized by gRNA for donor linearization
  misc_feature 24..571: note = 5' Homology Arm
  misc_feature 572..1285: note = mCLOVER sequence
  misc_feature 1286..1897: note = 3' Homology arm
  misc_feature 1898..1920: note = Sequence recognized by gRNA for donor linearization
ccatggagaccccgtcccagcggcacttccctctctttctccccgacccttttgcccacccactctccctccttggctctgccctctagcccagaaggtctgaggcaatgggggcaagcttggagccgacagtgctgagcaggcaggagccaagagaggggaagcttgagcctcacgcagttaggggtgcgctggagagggtggggcccgactccgccacaccccaacggtccttccccctcctcaccactcccgcccccacccccaatggatctgggactgcccctttaagagtagtggcccctcctcccttcagaggaggacctattagagcctttgccccggcgtcggtgactcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgcaacgcgggcagcgctgccaacctgccggccatggaggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggctaacttcaagatccgccacaacgttgaggacggcagcgtgcagctcgccgaccactaccagcagaacacccccatcggcgacggccccgtgctgctgcccgacaaccactacctgagccatcagtccgccctgagcaaagaccccaacgagaagcgcgatcacatggtcctgctggagttcgtgaccgccgccgggattacacatggcatggacgagctgtacaagaccccgtcccagcgacgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcggtgagttcgcccaggtggctgcgtgcctggcggggagtggagagggcggcgggccggcgcccctggccggccgcaggaagggagtgagagggcctggaggccgataactttgccatagtctcctccctccccggaactgcccccagcgggtgactggcagtgtcaaggggaattgtcaagacaggacagagagggaagtggtggtctctgggagagggtcggggaggatataaggaatggtgggggtatcagggacaagttgccatggagaccccgtcccagcgg

SEQ ID NO: 45 - DNA, 43 bp, synthetic construct
  source 1..43: mol_type = other DNA
aacctgccggccatggagaccccgtcccagcggcgcgccaccc